(19) United States
(12) Patent Application Publication
(10) Pub. No.: US 2007/0066924 A1
(43) Pub. Date: Mar. 22, 2007

(54) Supple tissue dressing assemblies, systems, and methods formed from softened hydrophilic polymer sponge structures such as chitosan

(75) Inventors: Lance David Hopman, Tigard, OR (US); Clinton Boyd Pepper, West Linn, OR (US); Dragos Horia Mihalache, Vancouver, WA (US); Michael S. Radovan, West Linn, OR (US); Simon J. McCarthy, Portland, OR (US)

Correspondence Address: RYAN KROMHOLZ & MANION, S.C., POST OFFICE BOX 26618, MILWAUKEE, WI, 53226, US

(73) Assignee: HemCon Medical Technologies, Inc.

(21) Appl. No.: 11/520,357

(22) Filed: Sep. 13, 2006

Related U.S. Application Data
(63) Continuation-in-part of application No. 11/202,558, filed on Aug. 12, 2005.
Application No. 11/202,558 is a continuation-in-part of application No. 11/020,365, filed on Dec. 23, 2004.

(51) Int. Cl. A61F 13/00

(52) U.S. Cl. 602/48

(57) ABSTRACT

Supple tissue dressing assemblies are formed from hydrophilic polymer sponge structures, such as chitosan. The supple tissue dressing assemblies are characterized by suppleness or multi-dimensional flexibility. The assemblies can be flexed, bent, folded, twisted, and even rolled upon itself before and during use, without creasing, cracking, fracturing, otherwise compromising the integrity and mechanical and/or therapeutic characteristics of the assemblies.

### RELATED APPLICATIONS

This application is a continuation-in-part of U.S. patent application Ser. No. 11/202,558, filed Aug. 12, 2005, and entitled “Tissue Dressing Assemblies, Systems, and Methods Formed from Hydrophilic Polymer Sponge Structures Such as Chitosan,” which is a continuation-in-part of U.S. patent application Ser. No. 11/020,365, filed Dec. 23, 2004, and entitled “Tissue Dressing Assemblies, Systems, and Methods Formed from Hydrophilic Polymer Sponge Structures Such as Chitosan,” which are each incorporated herein by reference.

### FIELD OF THE INVENTION

The invention is generally directed to tissue dressings applied on a site of tissue injury, or tissue trauma, or tissue access to ameliorate bleeding, fluid seepage or weeping, or other forms of fluid loss, as well as provide a protective covering over the site.

### BACKGROUND OF THE INVENTION

HemCon® Bandages made and sold by HemCon Medical Technologies Inc. (Portland, Oreg.) incorporate a chitosan sponge matrix having superior adhesive properties and resistance to dissolution in high blood flow, which make them well suited for stanching of severe arterial blood flow.

There always remains a need for improved hemostatic dressings that couple flexibility and ease of use with robustness and longevity required for resisting dissolution during use.

### SUMMARY OF THE INVENTION

The invention provides supple tissue dressing assemblies, systems and methods formed from hydrophilic polymer sponge structures, such as chitosan. The supple tissue dressing assemblies are characterized by suppleness or multi-dimensional flexibility. The assemblies can be flexed, bent, folded, twisted, and even rolled upon itself before and during use, without creasing, cracking, fracturing, otherwise compromising the integrity and mechanical and/or therapeutic characteristics of the assemblies. The supple tissue dressing assemblies can be densified, if desired, to increase their adhesion and cohesion strengths, as well as impart increased dissolution resistance in the presence of larger volumes of blood and fluids. The supple tissue dressing assemblies can also be further softened by mechanical manipulation, if desired, which lends enhanced flexibility and compliance.

According to one aspect of the invention, the supple tissue dressing assembly comprises a biocompatible hydrophilic polymer form. The form is softened by a mechanical softening device comprising a plurality of spaced apart upper and lower rolling surfaces defining between them an undulating path sized and arranged to knead the form along an axis.

The supple tissue dressing assemblies can be used, e.g., (i) to stanch, seal, or stabilize a site of tissue injury, tissue trauma, or tissue access; or (ii) to form an anti-microbial barrier; or (iii) to form an antiviral patch; or (iv) to intervene in a bleeding disorder; or (v) to release a therapeutic agent; or (vi) to treat a mucosal surface; or (vii) to dress a staph or MRSA infection site; or (viii) in various dental surgical procedures, or (ix) combinations thereof.

Other features and advantages of the invention shall be apparent based upon the accompanying description, drawings, and claims.

### DESCRIPTION OF THE DRAWINGS

FIG. 1 is a perspective view of a representative embodiment of a formed hydrophilic sponge material desirably comprising a chitosan matrix, which is sized and figured as a supple tissue dressing assembly.

FIG. 2 is a perspective view of the supple tissue dressing assembly shown in FIG. 1, after having been rolled upon itself for use by a caregiver.

FIG. 3 is a perspective view of another representative embodiment of a formed hydrophilic sponge material desirably comprising a chitosan matrix, which is sized and figured as a supple tissue dressing assembly.

FIG. 4 is a perspective view of the supple tissue dressing assembly shown in FIG. 4, being flexed in the hands of a caregiver.

FIG. 5 is a perspective view of the supple tissue dressing assembly, shown in roll form in FIG. 2, being unwrapped from the roll form, and then shaped, pushed, and/or stuffed into a wound track by a caregiver.

FIG. 6 is a perspective view of the supple tissue dressing assembly shown in FIG. 1 being cut or torn by a caregiver into smaller segments prior to use.

FIG. 7 is the segment of the supple tissue dressing assembly shown in FIG. 6 by shaped, pushed, and/or stuffed for topical application into a smaller wound track by a caregiver.

FIG. 8 is a perspective view of the supple tissue dressing assembly, shown in FIGS. 3 and 4, being applied to a dressing site by a caregiver.

FIG. 9 is a perspective view of a sealed pouch into which the supple tissue dressing assembly shown in roll form in FIG. 2 or the flat tissue dressing assembly shown in FIG. 3 is placed and sterilized prior to use by a caregiver.

FIGS. 10A and 10B are perspective views of the pouch shown in FIG. 6 being opened by a caregiver to gain access to the supple tissue dressing assembly for use.

FIG. 11 is a perspective view of the supple tissue dressing assembly shown in FIG. 1, after having been shaped, pushed, and/or stuffed into a wound track by a caregiver as shown in FIG. 3, being backed with a Kerlix™ roll or gauze for the purpose of applying pressure to the wound.

FIG. 12 is a perspective view of a caregiver wrapping gauze about the supple tissue dressing assembly shown in FIG. 11, after having been shaped, pushed, and/or stuffed into a wound track and pressure applied to stanch, seal, and/or stabilize a site of tissue injury.

FIG. 13 is a perspective view of two of the supple tissue dressing assemblies, shown in roll form in FIG. 2, being unwrapped from the roll form, and then shaped, pushed, and/or stuffed in a side-by-side relationship into a wound track by a caregiver.

FIGS. 14A and 14B are perspective views of representative molds in which a hydrophilic sponge material desirably comprising chitosan can be formed by freezing and freeze-drying to form the supple tissue dressing assembly shown, respectively, in FIG. 1 and FIG. 3.

FIGS. 15A and 15B are perspective views of a measured volume of chitosan solution being placed into the molds shown in FIGS. 14A and 14B prior to freezing.

FIG. 16 is a perspective view of a freezer in which the chitosan solution, after having been placed into a molds as shown in FIGS. 15A and 15B, is subjected to a prescribed freezing regime and subsequent freeze drying step.

FIGS. 17A and 17B are scanning electron microscope images (respectively at 30.0 kV×30 and 30.0 kV×100) of side sections of a desirable chitosan matrix (at room temperature without densification) that is formed as a result of the prescribed freezing regime and a subsequent freeze drying step within the freezer shown in FIG. 16, the freezing regime lowering the temperatures of the shelf, mold, chitosan solution, and air from room temperature to a freezing temperature at approximately the same rate (including a 30 minute freezing delay interval at 5° C.) to achieve a combined spherulitic and lamella nucleation of crystalline ice and subsequent phase separation that results in an inherently supple chitosan matrix structure.

FIG. 18 is a graph showing the phases of a prescribed freezing regime, including a freezing delay interval, that results in the creation of a desirable chitosan matrix structure of the type shown in FIGS. 17A and 17B.

FIGS. 19A and 19B are perspective views of the removal of a supple chitosan matrix structure from the molds shown in FIGS. 15A and 15B after undergoing the freezing regime shown in FIG. 18 as well as a subsequent prescribed freeze-drying process.

FIG. 20 is a perspective view showing flexure of the supple, chitosan matrix structure after removal from the mold, as shown in FIG. 19A.

FIGS. 21A, 21B, and 21C show the subsequent densification of the supple, chitosan matrix structure shown in FIG. 20, to create a supple, densified chitosan matrix structure.

FIG. 22 is a perspective view of an oven which preconditions the supple, densified chitsan matrix structure shown in FIG. 21C.

FIG. 23 is a perspective view of a softening machine, which subjects the supple, densified and preconditioned chitsan matrix structure (FIGS. 21C and 22) to gentle, systematic mechanical softening along its longitudinal axis (length direction), which improves its inherent suppleness and compliance.

FIG. 24 is a side view of the array of upper and lower rollers that form a part of the softening machine shown in FIG. 23.

FIG. 25 is a more diagrammatic, side view of the array of upper and lower rollers shown in FIG. 24, with the supple, densified chitsan matrix structure traversing the serpentine path between the upper and lower rollers.

FIG. 26 is a side view of an optional second softening array, which can be arranged either before or after the first array of upper and lower rollers shown in FIGS. 24 and 25, to compress or knead the supple densified chitosan matrix structure along its transverse axis (width direction).

FIG. 27 is a more diagrammatic, side view of the second softening array shown in FIG. 26, with the supple, densified chitsan matrix structure traversing the serpentine path between the upper and lower wheels of the second softening array.

FIGS. 28A, 28B, and 28C are perspective views of an alternative embodiment of a softening tool that softens along the width direction of the matrix.

FIGS. 29A and 29B are, respectively, perspective exploded and assembled views of alternative supple, densified tissue dressing assemblies that can be created in different sizes and shapes using the manufacturing steps shown in FIGS. 14 to 28, and which can, if desired, include a backing material.

FIG. 30 is a graph comparing the flexibility of a supple densified tissue dressing assembly to the flexibility of a state of the art tissue dressing matrix.

### DETAILED DESCRIPTION

Although the disclosure hereof is detailed and exact to enable those skilled in the art to practice the invention, the physical embodiments herein disclosed merely exemplify the invention, which may be embodied in other specific structure. While the preferred embodiment has been described, the details may be changed without departing from the invention, which is defined by the claims.

### I. SUPPLE TISSUE DRESSING ASSEMBLY

A. Overview

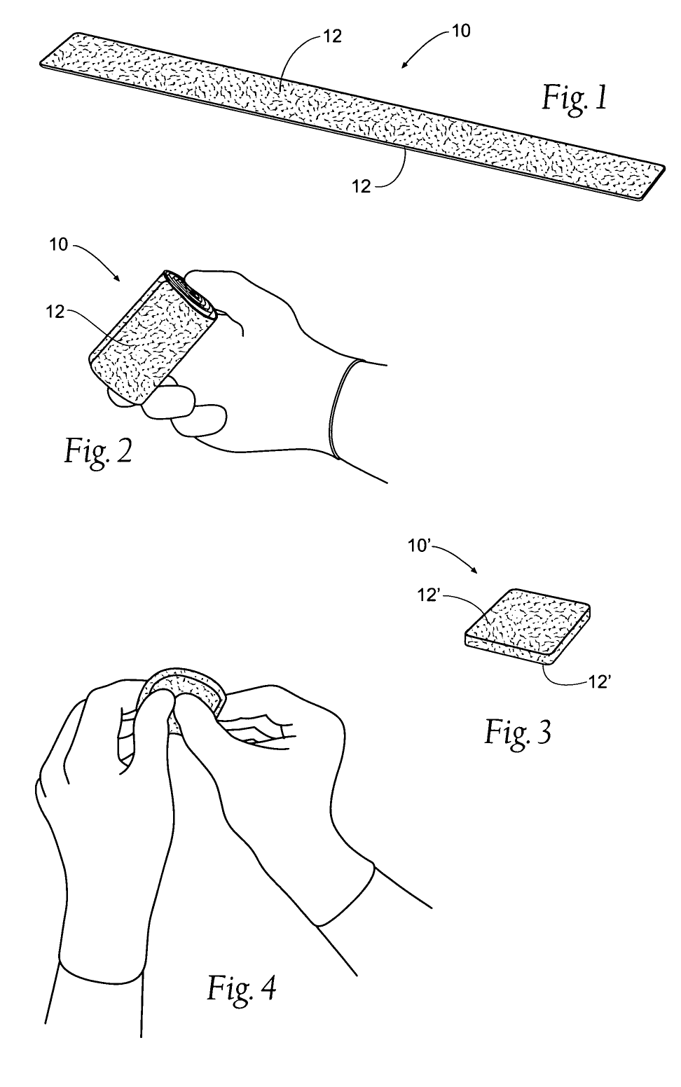

FIGS. 1 and 2 show a representative embodiment of a supple tissue dressing assembly 10 that embodies features of the invention. As shown, the supple tissue dressing assembly 10 comprises a relatively thin and supple tissue dressing matrix 12 (shown FIG. 1) comprising a hydrophilic polymer that can be characterized as a supple sponge structure. As shown in FIGS. 1 and 2, and as will be described in greater detail later, the tissue dressing matrix 12 is formed by subjecting a solution of the hydrophilic polymer to a prescribed freezing regime followed by freeze drying (lyophilization), which creates a unique dry supple sponge structure. In the embodiment shown in FIGS. 1 and 2, the dry supple sponge structure forming the matrix 12 is further mechanically compressed to a reduced thickness (e.g., from about 4 mm to 0.25 mm, and desirably about 0.9 mm) and an increased density (e.g., from about 0.1 g/cm³ to about 0.5 g/cm3, and most desirably about 0.2 g/cm³).

FIGS. 3 and 4 show another representative embodiment of a supple tissue dressing assembly 10′. As shown in FIG. 3, the supple tissue dressing assembly 10′ comprises a matrix 12′ possessing the same unique supple sponge structure of the assembly 10 shown in FIGS. 1 and 2, which is formed in generally the same manner by a prescribed freezing regime and freeze drying. In the embodiment shown in FIG. 3, however, the dry supple sponge structure forming the matrix 12′ is not mechanically compressed and densified, and is therefore thicker (e.g., about 1 mm to about 8 mm thick) and less dense (e.g., a density of about 0.03 g/cm³, more or less) than the matrix 12 shown in FIGS. 1 and 2.

The unique underlying dry sponge structure that comprises both tissue dressing matrixes 12 and 12′ is characterized by its suppleness or multi-dimensional flexibility. Before densification (as FIG. 4 shows) or after densification (as FIG. 2 shows), the dry matrix 12 and 12′ can be flexed, bent, folded, twisted, and even rolled upon itself before and during use, without creasing, cracking, fracturing, otherwise compromising the integrity and mechanical and/or therapeutic characteristics of the matrix 12 and 12′. The unique underlying dry sponge structure that comprises both tissue dressing matrixes 12 and 12′ (either after or before densification) can also be characterized by a suppleness or multi-dimensional flexibility in terms of a Gurley stiffness value (in units of milligrams) (when dry) of not greater than about 5000 (using a Gurley Stiffness Tester Model 4171D manufactured by Gurley Precision Instruments of Troy, N.Y., and Gurley ASTM D6125-97). It is believed that a dry sponge structure having a Gurley stiffness value (in units of milligrams) greater than about 5000 do not possess the requisite suppleness or multi-dimensional flexibility to be flexed, bent, folded, twisted, and even rolled upon itself before and during use, without creasing, cracking, fracturing, otherwise compromising the integrity and mechanical and/or therapeutic characteristics of the matrix 12 and 12′. Desirably, the unique underlying dry sponge structure that comprises both tissue dressing matrixes 12 and 12′ (either after or before densification) is characterized by a suppleness or multi-dimensional flexibility in terms of a Gurley stiffness value (in units of milligrams) (when dry) of not greater than about 2500, and most desirably, at or about 1000.

The underlying dry sponge structure that comprises both tissue dressing matrixes 12 and 12′ can also be characterized when dry by the unique combination of a clinically effective tensile strength (integrity) with the suppleness as previously described. This unique combination of physical attributes that the underlying dry sponge structure of the matrix 12 or 12′ provides, can be expressed in terms of a ratio between the Gurley stiffness value (in units of milligrams) (as determined when dry by using a Gurley Stiffness Tester Model 4171D manufactured by Gurley Precision Instruments of Troy, N.Y., and Gurley ASTM D6125-97) and tensile strength (expressed in units of Newtons) (as determined when dry by an Instron™ Device and ASTM Test Method D412 (Method A, Section 12)). This ratio will in shorthand be called the dry suppleness-to-strength ratio. The matrix 12 and 12′ can provide a dry suppleness-to-strength ratio value of not greater than about 210, which makes possible a relatively high clinically useful tensile strength (e.g., 10 Newtons) with a supple structure having relatively low Gurley stiffness value (e.g., 2000 Gurley Units), particularly when the matrix 12 is used in densified form.

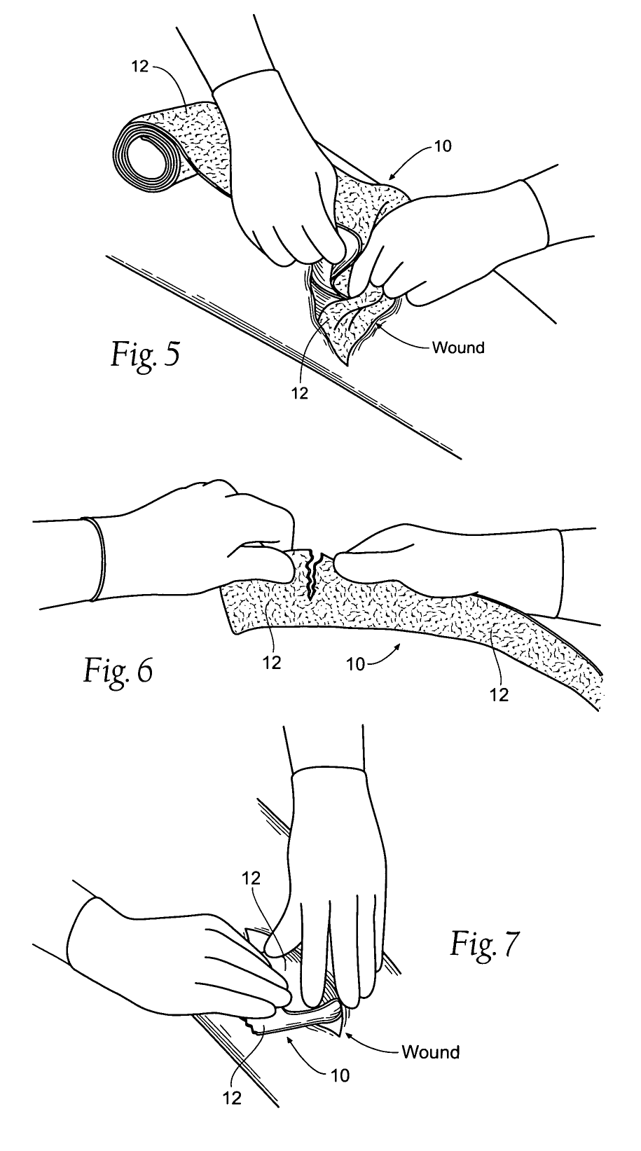

In densified form (as shown in FIG. 5), the dry supple tissue dressing assembly 10 can be readily sized and configured to be unwrapped from a roll form, and then shaped, pushed, and/or stuffed into a wound track. In densified form (as shown in FIGS. 6 and 7), the dry tissue dressing matrix 12 can be readily cut or torn into smaller segments (FIG. 6) for topical application upon or insertion within a smaller wound (FIG. 7). For a smaller wound (as FIG. 7 shows), once torn or cut from the roll, a segment of the dry tissue dressing matrix 12 can be readily folded into a “C” shape or another configuration to facilitate its insertion into a wound track. The densification of the dry matrix 12 imparts increased dissolution resistance in the presence of larger volumes of blood and fluids.

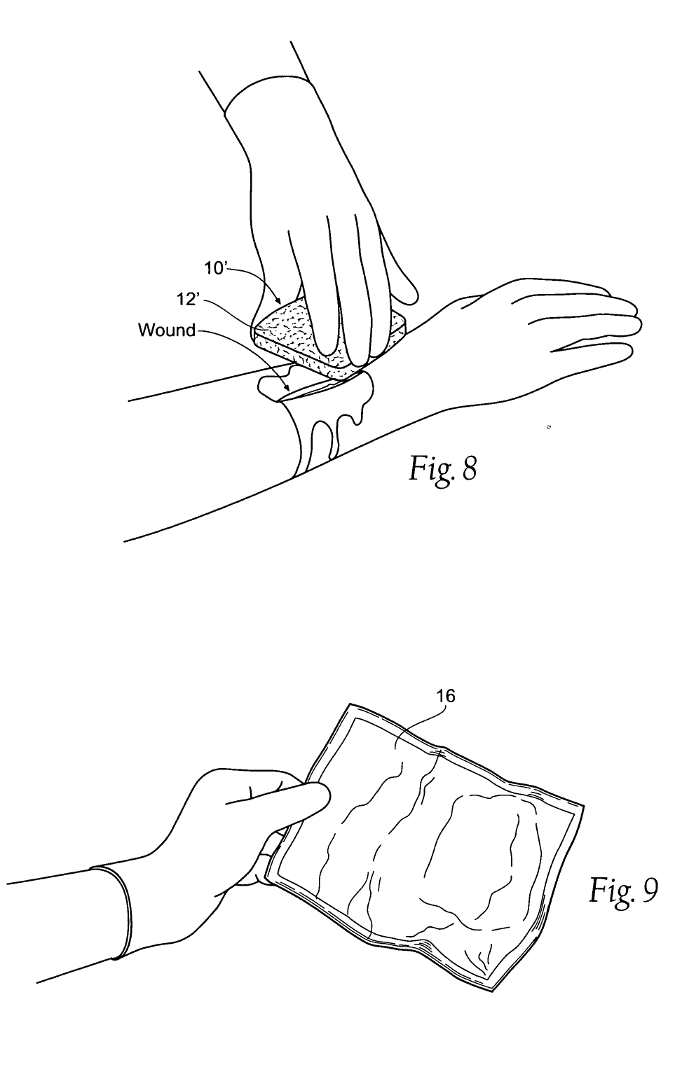

Without densification (as shown in FIG. 8), the dry supple tissue dressing assembly 10′ can be sized and configured with smaller, preformed dimensions for topical application for, e.g., low bleeding hemostasis and/or antibacterial/antiviral wound dressing applications. Of course, a densified matrix 12 can also be used for such applications, too. Also, without densification, the dry tissue dressing matrix 12′ can be cut or torn as desired into even smaller segments or into different shapes to conform to the topology of the application site.

In the embodiments shown in FIGS. 1 to 8, the hydrophilic polymer is exposed both sides of the dry supple tissue dressing matrix 12 and 12′. The hydrophilic polymer is elected to comprise a material that adheres to tissue in the presence of blood, or body fluids, or moisture. The supple tissue dressing assembly 10 or 10′ can thus be used to stanch, seal, and/or stabilize a site of tissue injury, or tissue trauma, or tissue access (e.g., a catheter or feeding tube) against bleeding, fluid seepage or weeping, or other forms of fluid loss. The tissue site treated can comprise, e.g., arterial entrance/entry wound, or a tissue puncture, or a catheter access site, or a burn, or a suture, or an open tooth socket. The supple tissue dressing assembly 10 can also desirably form an anti-bacterial and/or anti-microbial and/or anti-viral protective barrier at or surrounding the tissue treatment site.

The particular size, shape, and configuration of the supple tissue dressing matrix 12 and 12′ can, of course, vary according to its intended use. As will be described in greater detail later, the supple tissue dressing matrix 12 and 12′ is shaped by a mold during manufacture, either into the elongated and rectilinear form shown in FIG. 1 or in the smaller form shown in FIG. 3.

In a representative embodiment (shown in FIGS. 1 and 2), the elongated tissue dressing matrix 12 can be formed, with mechanical compression and densification, with an overall length of about 28 inches (711 mm), a width of about 3 inches (76 mm), and a thickness of about 0.35 inch (0.9 mm), more or less. The thickness of a densified matrix 12 can range from about 0.25 mm to about 4 mm. As just noted, the elongated tissue dressing matrix 12 has the flexibility to be bent, flexed, twisted or rolled upon itself, without creasing, cracking, or fracture. As shown in FIG. 2, elongated tissue dressing matrix 12 can be manually rolled tightly upon itself, to form a roll that can be as small as about 1.5 inches (38 mm) in diameter, depending upon how tightly rolled the matrix is. Due to its suppleness, the initial elongated form of the tissue dressing matrix 12 can be rolled upon itself without fracture into the roll form shown in FIG. 2, which has a diameter that less than either the width or the length of the initial elongated form.

In another representative embodiment (shown in FIGS. 3 and 4) the matrix 12′ can be formed, without compression or densification, with smaller dimension, e.g., 2 inches (51 mm) by 2 inches (51 mm) by 0.16 inch (4 mm) or even smaller (e.g., for dental applications), e.g., 10 mm×12 mm and about 4 mm thick, more or less. The thickness for an undensified matrix 12′ can range between about 1 mm to 8 mm. The smaller tissue dressing matrix 12′ also has the flexibility to be bent, flexed, twisted or rolled upon itself, without creasing, cracking, or fracture.

Of course, diverse other sizes and shapes—e.g., square, round, oval, or a composite or complex combination thereof—are possible. As previously described, the shape, size, and configuration of assembly 10 can be further altered after manufacture by cutting, bending, molding, folding, or twisting either during use or in advance of use.

1. The Tissue Dressing Matrix

The biocompatible material selected for the matrix 12 and 12′ desirably reacts in the presence of blood, body fluid, or moisture to become a strong adhesive or glue. Desirably, the selected biocompatible material also possesses other beneficial attributes, for example, anti-bacterial and/or anti-microbial anti-viral characteristics, and/or characteristics that accelerate or otherwise enhance the body's defensive reaction to injury.

The tissue dressing matrix 12 and 12′ may comprise a hydrophilic polymer form, such as a polyacrylate, an alginate, chitosan, a hydrophilic polyamine, a chitosan derivative, polylysine, polyethylene imine, xanthan, carrageenan, quaternary ammonium polymer, chondroitin sulfate, a starch, a modified cellulosic polymer, a dextran, hyaluronan or combinations thereof. The starch may be of amylase, amylopectin and a combination of amylopectin and amylase.

The biocompatible material of the matrix 12 and 12′ preferably comprises the non-mammalian material poly [β-(1→4)-2-amino-2-deoxy-D-glucopyranose, which is more commonly referred to as chitosan.

The chitosan matrix 12 and 12′ presents a robust, permeable, high specific, positively charged surface. The positively charged surface creates a highly reactive surface for red blood cell and platelet interaction. Red blood cell membranes are negatively charged, and they are attracted to the chitosan matrix 12 and 12′. The cellular membranes fuse to chitosan matrix 12 and 12′ upon contact. A clot can be formed very quickly, circumventing immediate need for clotting proteins that are normally required for hemostasis. For this reason, the chitosan matrix 12 and 12′ is effective for both normal as well as anti-coagulated individuals, and as well as persons having a coagulation disorder like hemophilia. The chitosan matrix 12 and 12′ also binds bacteria, endotoxins, and microbes, and can kill bacteria, microbes, and/or viral agents on contact.

As will be described in greater detail later, the hydrophilic polymer matrix 12 and 12′ is created by subjecting a solution of the chitosan hydrophilic polymer to phase separation by a controlled freezing process, followed by a controlled water removal step by freeze-drying or lyophilization. As will be described in greater detail later, the parameters of the freezing and lyophilization processes are controlled to create a dry supple sponge-like structure for the chitosan matrix 12 and 12′. Due to its inherent suppleness, the dry chitosan matrix 12 and 12′ is not stiff or brittle. It possesses an inherent capability for flexure and/or twisting without compromising its structural integrity and mechanical and therapeutic properties. As will also be described later, the inherent suppleness of dry chitosan matrix 12 and 12′ can also be further enhanced by a mechanical softening process.

As will also be described later, the density of the particular dry chitosan structure of the matrix 12 following freezing and freeze drying can be increased by a mechanical densification process. The mechanical densification process imparts enhanced adhesion strength, cohesion strength and dissolution resistance of the matrix 12 in the presence of blood or body fluids.

2. The Pouch

As FIG. 9 shows, before use, the tissue dressing assembly 10 and 10′ is desirably vacuum packaged in roll form with low moisture content, preferably 5% moisture or less, in an air-tight heat sealed foil-lined pouch 16. The tissue dressing assembly 10 and 10′ is subsequently terminally sterilized within the pouch 16 by use of gamma irradiation.

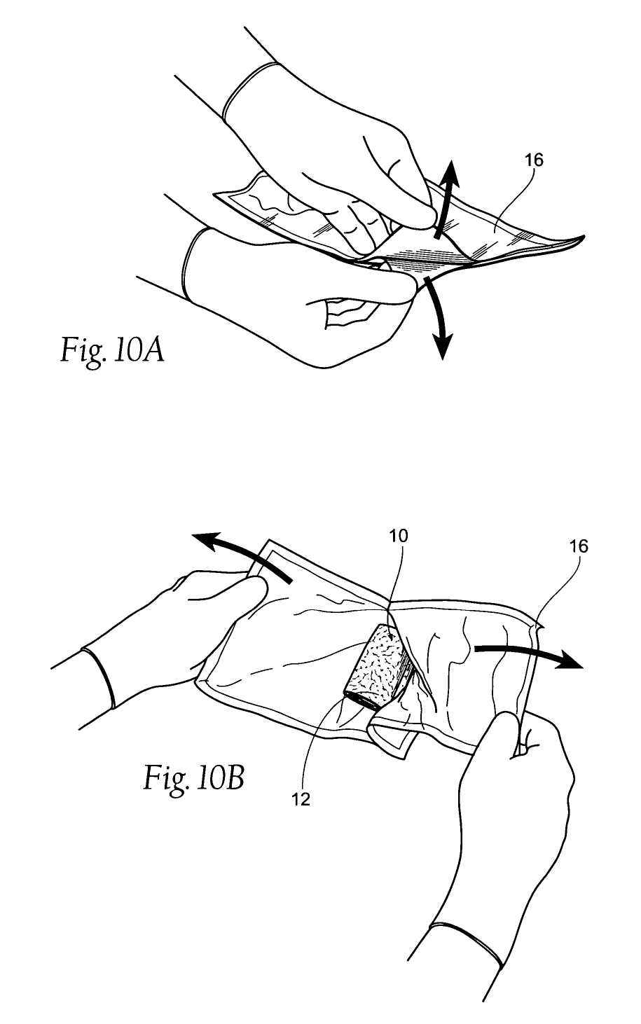

The pouch 16 is configured to be peeled opened by the caregiver (see FIGS. 10A and 10B) at the instant of use. The pouch 16 provides peel away access to the tissue dressing assembly 10 and 10′ along one end (the roll-form densified tissue dressing assembly 10 is shown in FIG. 10B for purposes of illustration). The opposing edges of the pouch 16 are grasped and pulled apart (FIG. 10A) to expose the tissue dressing pad assembly 10 and 10′ for use. As the pouch 16 begins to open (FIG. 10B), care should be taken so that the assembly 10 and 10′ does not drop to the ground.

B. Use of the Supple Tissue Dressing Assembly

Once removed from the pouch 16 (see FIGS. 2 and 4), the tissue dressing assembly 10 and 10′ is immediately ready to be adhered to the targeted tissue site. It needs no pre-application manipulation to promote adherence. For example, there is no need to peel away a protective material to expose an adhesive surface for use. The adhesive surface forms in situ, because the chitosan matrix 12 and 12′ itself exhibits strong adhesive properties once in contact with blood, fluid, or moisture.

Desirably, the tissue dressing assembly 10 and 10′ is applied to the injury site immediately upon opening the pouch 16. FIG. 5 shows the densified assembly 10 being applied for treating an arterial and/or venous bleeding injury. The chitosan matrix 12 is active on both sides of the assembly 10. The entire assembly 10 will become sticky when it is placed into contact with blood. Desirably, the assembly 10 is handled quickly and pushed aggressively into the wound track (as FIG. 5 shows). The assembly 10 is desirably placed directly on the source of bleeding, i.e., the area where the blood vessel damage has actually occurred. Desirably, once applied, the assembly 10 is not re-positioned.

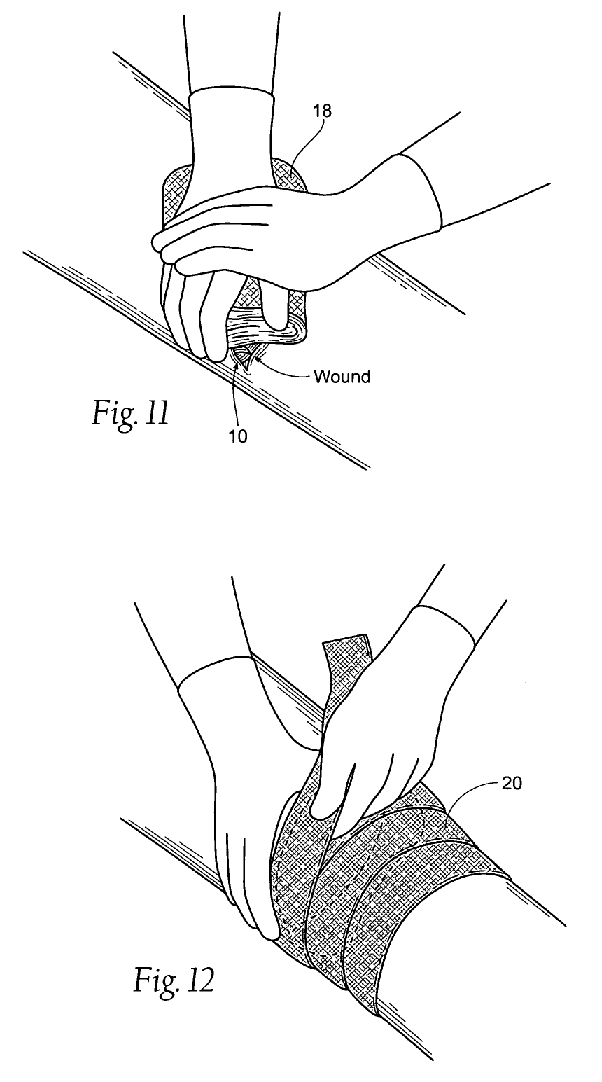

With the densified assembly 10 inserted in the wound track (see FIG. 11), the assembly 10 can be backed with Kerlix™ roll or gauze 18, and pressure applied to the wound. Desirably, pressure is applied on the assembly 10 for at least two minutes, or until the assembly 10 adheres and the blood is controlled. Firm pressure is applied, to allow the natural adhesive activity of the chitosan matrix 12 to develop. The adhesive strength of the chitosan matrix 12 will increase with duration of applied pressure, up to about five minutes. Pressure applied evenly across the assembly 10 during this time will provide more uniform adhesion and wound sealing.

Once pressure has been applied for the requisite time, e.g., two to five minutes, and/or control of the bleeding has been accomplished with good dressing adhesion and coverage of the wound or tissue site, a second conventional dressing 20 (e.g., gauze) is desirably applied (see FIG. 12) to secure the dressing to provide a clean barrier for the wound. If the wound is to be subsequently submersed underwater, a water tight covering should be applied to prevent the dressing assembly 10 from becoming over-hydrated.

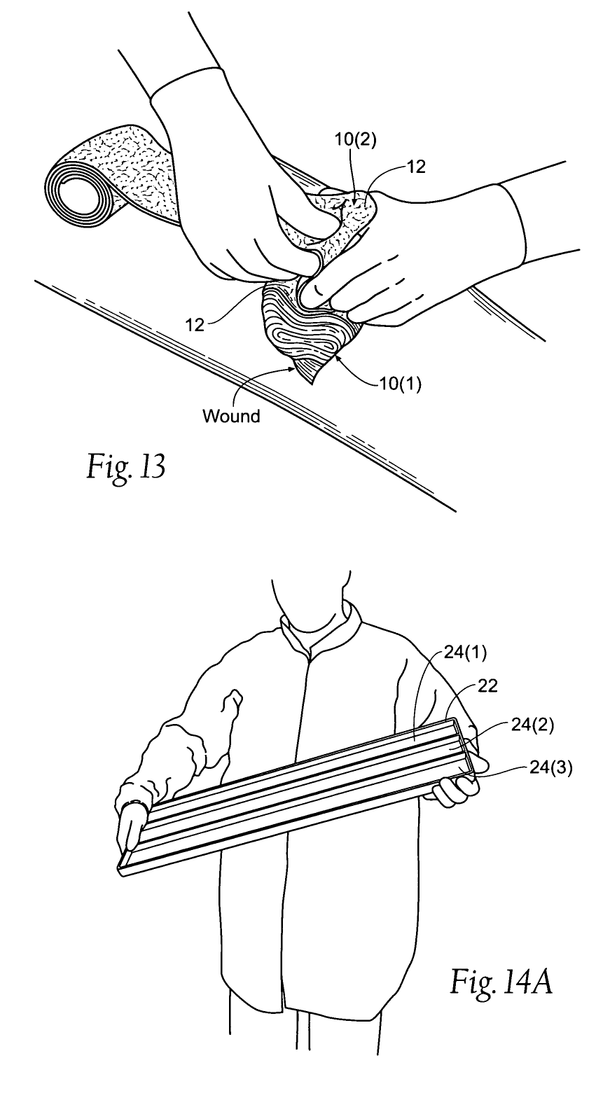

Due to unique mechanical and adhesive characteristics, two or more densified dressing assemblies 10(1) and 10(2) (see FIG. 13) can be applied side-by-side, if needed, to occupy the wound or tissue site. The chitosan matrix 12 of one assembly 10 will adhere to the chitosan matrix 12 of an adjacent assembly 10.

The smaller, uncompressed dressing assembly 10′ shown in FIGS. 3 and 4 can also be appropriately applied to an intended dressing site. It, too, will become sticky when it is placed into contact with blood or body fluids, and will adhere as pressure is applied. When good dressing adhesion and coverage of the dressing site are achieved, a second conventional dressing (e.g., gauze) can be applied to secure the dressing 10′ and to provide a clean barrier for the wound.

As previously described, and as shown in FIGS. 6 and 7, the assembly 10 (or assembly 10′) can also be torn or cut on site to match the size of the wound or tissue site. Smaller, patch pieces of an assembly 10 or 10′ can also be cut to size on site, and fitted and adhered to the periphery of another assembly 10 or 10′ to best approximate the topology and morphology of the treatment site.

The supple assembly 10 or 10′ accommodates layering, compaction, and/or rolling—i.e., “stuffing” (as FIG. 5 shows for the densified assembly 10)—of the chitosan matrix 12 (or matrix 12′) within a wound site using pressure to further reinforce the overall structure against strong arterial and venous bleeding. By stuffing the supple densified assembly 10 over itself, as FIG. 5 shows, the interaction of the blood with the chitosan provides advantages for the application when the wounds are particularly deep or otherwise apparently inaccessible. The stuffing of the supple assembly 10 into a bleeding wound and its compression on itself provide for a highly adhesive, insoluble and highly conforming bandage form.

The assembly 10 and 10′ is intended to temporarily control severe bleeding. The assembly 10 can, when desired, be peeled away from the wound and will generally separate from the wound in a single, intact dressing. In some cases, residual chitosan gel may remain, and this can be removed using saline or water with gentle abrasion and a gauze dressing. Chitosan is biodegradable within the body and is broken down into glucosamine, a benign substance. Still, it is desirable in the case of temporary dressings, that efforts should be made to remove all portions of chitosan from the wound at the time of definitive repair.

C. Manufacture of the Chitosan Matrix

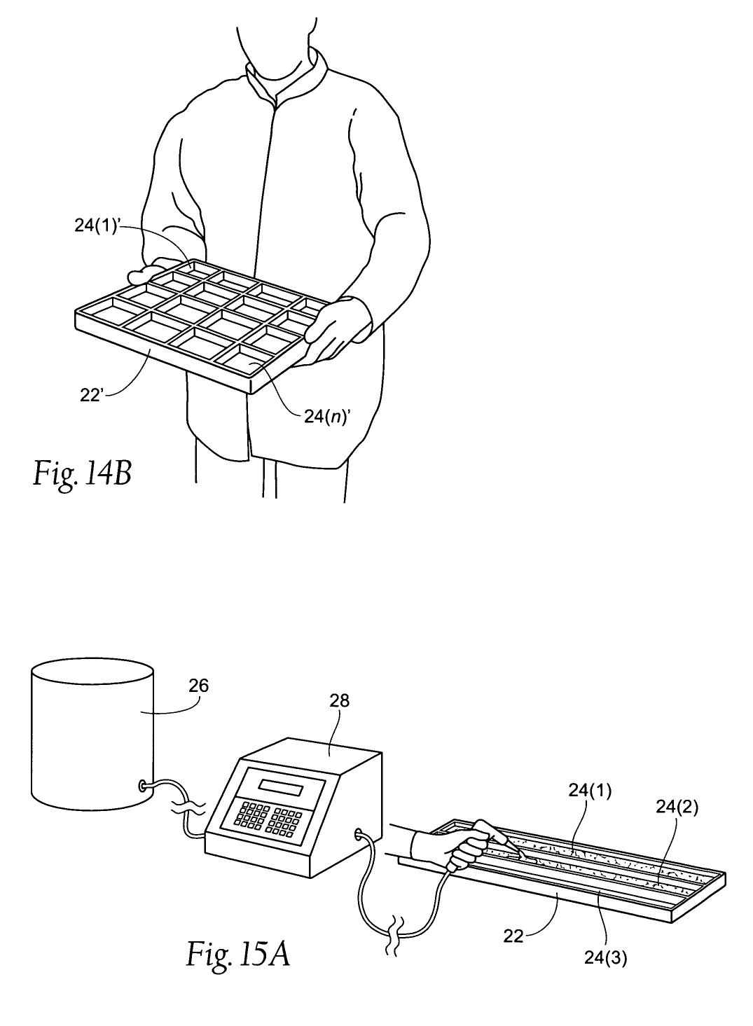

With reference to FIGS. 14A/14B to 20, a desirable methodology for making the matrix 12 or 12′ will now be described. It should be realized, of course, that other/methodologies can be used.

1. Preparation of a Chitosan Solution

In a preferred embodiment, the matrix 12 and 12′ comprises poly [β-(1→4)-2-amino-2-deoxy-D-glucopyranose, commonly referred to as chitosan. The chitosan selected for the matrix 12 and 12′ preferably has a weight average molecular weight of at least about 100 kDa, and more preferably, of at least about 150 kDa. Most preferably, the chitosan has a weight average molecular weight of at least about 300 kDa.

The chitosan used to prepare the chitosan solution preferably has a fractional degree of deacetylation greater than 0.78 but less than 0.97. Most preferably the chitosan has a fractional degree of deacetylation greater than 0.85 but less than 0.95. Preferably the chitosan selected for processing into the matrix has a viscosity at 25° C. in a 1%(w/w) solution of 1% (w/w) acetic acid (AA) with spindle LVI at 30 rpm, which is about 100 centipoise to about 2000 centipoise. More preferably, the chitosan has viscosity at 25° C. in a 1% (w/w) solution of 1% (w/w) acetic acid (AA) with spindle LVI at 30 rpm, which is about 125 centipoise to about 1000 centipoise. Most preferably, the chitosan has viscosity at 25° C. in a 1% (w/w) solution of 1% (w/w) acetic acid (AA) with spindle LVI at 30 rpm, which is about 400 centipoise to about 800 centipoise.

In forming the matrix 12 and 12′, the chitosan is desirably placed into solution with an acid, such as glutamic acid, lactic acid, formic acid, hydrochloric acid, glycolic acid, and/or acetic acid. Among these, hydrochloric acid and acetic acid are most preferred, because chitosan acetate salt and chitosan chloride salt resist dissolution in blood whereas chitosan lactate salt and chitosan glutamate salt do not. Larger molecular weight (Mw) anions disrupt the para-crystalline structure of the chitosan salt, causing a plasticization effect in the structure (enhanced flexibility). Undesirably, they also provide for rapid dissolution of these larger Mw anion salts in blood.

The chitosan solution is preferably prepared at 25° C. by addition of water to solid chitosan flake or powder and the solid dispersed in the liquid by agitation, stirring or shaking. On dispersion of the chitosan in the liquid, the acid component is added and mixed through the dispersion to cause dissolution of the chitosan solid. The rate of dissolution will depend on the temperature of the solution, the molecular weight of the chitosan and the level of agitation. Preferably the dissolution step is performed within a closed tank reactor with agitating blades or a closed rotating vessel. This ensures homogeneous dissolution of the chitosan and no opportunity for high viscosity residue to be trapped on the side of the vessel. Preferably the chitosan solution percentage (w/w) is greater than 0.5% chitosan and less than 2.7% chitosan. More preferably the chitosan solution percentage (w/w) is greater than 1% chitosan and less than 2.3% chitosan. Most preferably the chitosan solution percentage is greater than 1.5% chitosan and less than 2.1% chitosan. Preferably the acid used is acetic acid. Preferably the acetic acid is added to the solution to provide for an acetic acid solution percentage (w/w) at more than 0.8% and less than 4%. More preferably the acetic acid is added to the solution to provide for an acetic acid solution percentage (w/w) at more than 1.5% (w/w) and less than 2.5%.

2. Degassing the Aqueous Chitosan Solution

Preferably, the chitosan biomaterial is degassed of general atmospheric gases. Typically, degassing is removing sufficient residual gas from the chitosan biomaterial so that, on undergoing a subsequent freezing operation, the gas does not escape and form unwanted large voids or large trapped gas bubbles in the subject wound dressing product. The degassing step may be performed by heating a chitosan biomaterial, typically in the form of a solution, and then applying a vacuum thereto. For example, degassing can be performed by heating a chitosan solution to about 45° C. immediately prior to applying vacuum at about 500 mTorr for about 5 minutes while agitating the solution.

In one embodiment, certain gases can be added back into the solution to controlled partial pressures after initial degassing. Such gases would include but are not limited to argon, nitrogen and helium. An advantage of this step is that solutions containing partial pressures of these gases form micro-voids on freezing. The microvoid is then carried through the sponge as the ice-front advances. This leaves a well defined and controlled channel that aids sponge pore interconnectivity.

3. Freezing the Aqueous Chitosan Solution

The form producing steps for the chitosan matrix 12 and 12′ are typically carried out from the solution. The form producing steps can he accomplished employing techniques such as freezing (to cause phase separation), non-solvent die extrusion (to produce a filament), electro-spinning (to produce a filament), phase inversion and precipitation with a non-solvent (as is typically used to produce dialysis and filter membranes) or solution coating onto a preformed sponge-like or woven product.

In a preferred embodiment, the chitosan biomaterial—now in acid solution and degassed, as described above—is subjected to a form producing step that includes a controlled freezing process. The controlled freezing process is carried out by cooling the chitosan biomaterial solution within a mold 22 or 22′.

The mold 22 or 22′ can be variously constructed. As shown in FIG. 14A, the mold 22 for forming the elongated matrix 12 (FIGS. 1 and 2) can be made from a metallic material, e.g., Mic 6 aluminum, although other metallic materials and alloys can be used, such as iron, nickel, silver, copper, titanium, titanium alloy, vanadium, molybdenum, gold, rhodium, palladium, platinum and/or combinations thereof.

In a representative embodiment for creating a matrix 12 like that shown in FIGS. 1 and 2, the mold 22 measures overall 30 inches by 9.8 inches, and is compartmentalized into three mold chambers 24(1), 24(2), and 24(3), each 3 inches in width and 0.051 inch in depth. The mold chambers 24(1), 24(2), and 24(3) are desirably coated with a thin, permanently-bound, fluorinated release coating formed from polytetrafluoroethylene (Teflon), fluorinated ethylene polymer (FEP), or other fluorinated polymeric materials.

As FIG. 14B shows, the mold 22′ for forming the smaller matrix 12′ (FIGS. 3 and 4) can be made from a plastic material compartmentalized into multiple small wells or chambers 24(1)′ to 24(n)′ for forming multiples of assemblies 10′ at one time.

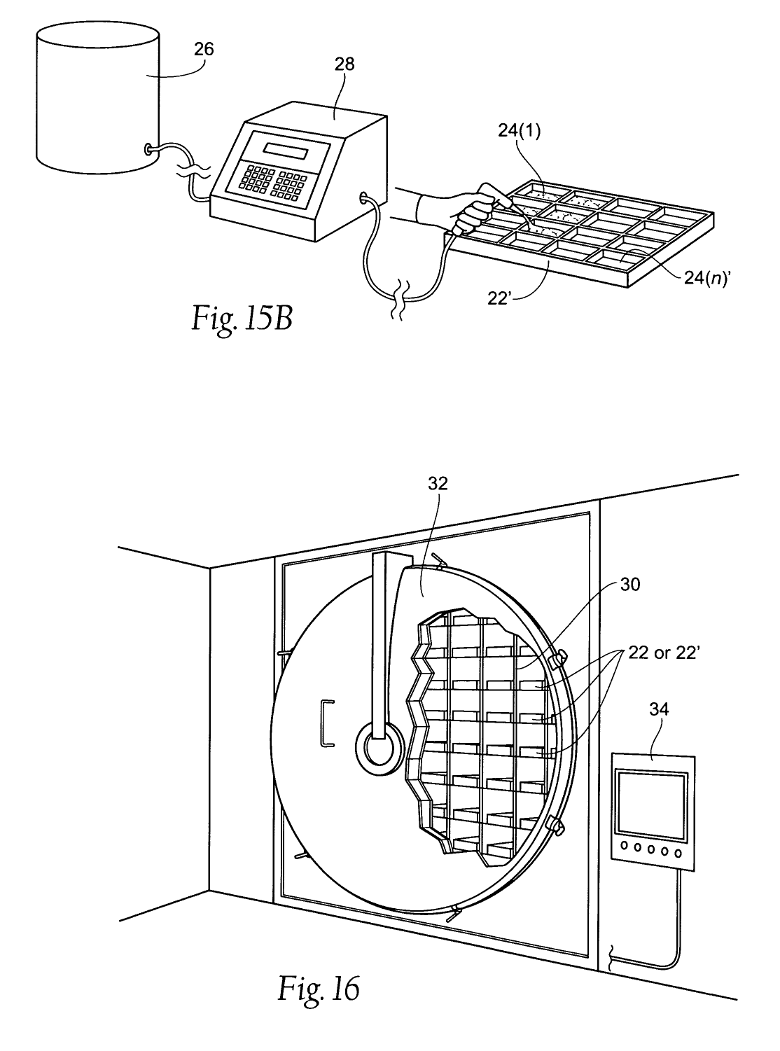

As FIGS. 15A and 15B show, a preselected volume of the chitosan biomaterial solution is conveyed from a source 26 into each mold chamber 24(1), 24(2), and 24(3) or 24(1)′ to 24(n)′ using, e.g., a positive displacement pump 28. Given the mold dimensions disclosed above for creating the eleongated matrix 10 (FIGS. 1 and 2), in a representative embodiment, 450 gr+/−13 of chitosan biomaterial solution is conveyed into each mold chamber 24(1), 24(2), and 24(3). Adding a lesser volume of the chitosan biomaterial solution will result in a matrix that, after molding, possesses a thinner cross section and therefore an ultimately thinner finished matrix 12 and 12′.

The mold 22 or 22′ and chitosan biomaterial solution are then located on flat stainless-steel heating/cooling shelves 30 within a freeze dryer 32 (FIG. 16). The flat base of each mold chamber 24(1), 24(2), and 24(3) or 24(1)′ to 24(n)′ is placed in close thermal contact with the flat stainless-steel heating/cooling surface of the shelf 30. A microprocessor controller 34 carries out the prescribed steps of the freezing process control algorithm.

Within the freezer 32, under the control of the controller 34, the temperature of the chitosan biomaterial solution is ultimately lowered from room temperature (e.g., about 20° C.) to a final temperature well below the freezing point (e.g., minus 40° C.). The chitosan biomaterial solution within each mold chamber 24(1), 24(2), and 24(3) or 24(1)′ to 24(n)′ loses heat uniformly through the shelf cooling surface and freezes. In this process, the chitosan biomaterial solution undergoes phase separation, which begins to form the desired structure of the matrix.

In a preferred embodiment, during the downward transition in temperatures from room temperature to the final freezing temperature, under the control of the controller 34, the freezing process desirably starts by equalizing the temperatures of the shelf, mold, biomaterial solution, and surrounding air at room temperature and then lowers the temperature of the shelf, mold, biomaterial solution, and air at approximately the same rate to achieve uniform nucleation during phase separation. It is believed that the desired cooling rate to achieve uniform nucleation is less than about 0.5° C./min. It is to be appreciated that the cooling rate is a negative number, because the temperature is dropping from room temperature to a colder freezing temperature. As expressed above, a cooling rate of 1.0° C./min is considered a greater negative rate and therefore not less than a cooling rate of 0.5° C./min. Conversely, a cooling rate of 0.3° C./min is considered a lesser negative rate and therefore is less than 0.5° C./min.

There are various ways for achieving this desired cooling rate and uniformity of temperature conditions among the shelf, mold, biomaterial solution, and air, depending upon the mechanical and operational characteristics and capabilities of the particular freeze dryer 32, e.g., its compressor capability (affecting the cooling rate) and heat flow homogeneity of the cooling chamber.

In a representative embodiment, the desired cooling rate and uniformity of temperature conditions is achieved by including a delay interval. During the delay interval, the controller 34 commands an intermediate temperature condition at a prescribed magnitude above the freezing point, which is held for a prescribed period of time before dropping the temperature to the final freezing temperature.

It has been discovered that imposing a prescribed delay interval in the freezing regime, or otherwise lowering the shelf, mold, biomaterial solution, and air temperature at approximately the same desired cooling rate, results in a supple chitosan sponge structure that is less stiff and brittle, and more readily accommodates flexure without fracturing the sponge structure. In comparison, it has been observed that a freezing regime that transitions temperatures from room temperature to a temperature well below the freezing point, without imposing a delay interval at an intermediate temperature condition above the freezing point, or otherwise lowering the shelf, mold, biomaterial solution, and air temperature at approximately the same desired cooling rate, results in a chitosan sponge structure that is more stiff and brittle, and therefore less able to accommodate extreme flexure without fracturing.

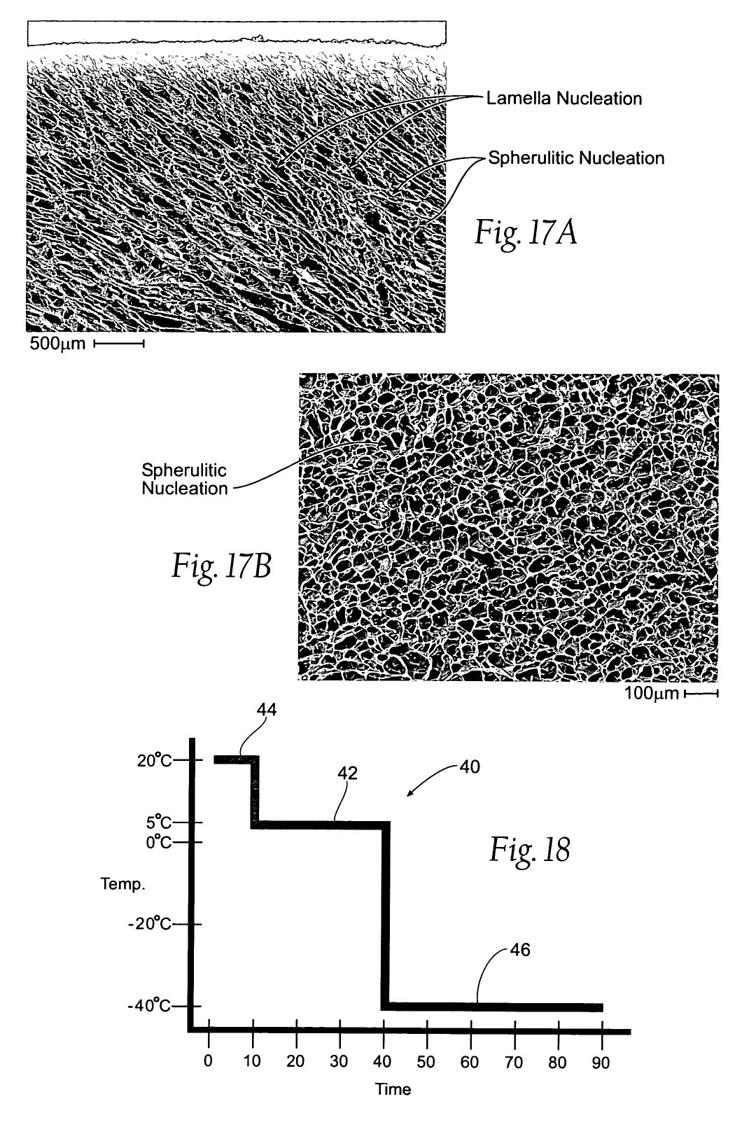

The delay interval produces a preferred structure for the chitosan matrix 12 of a type shown in FIGS. 17A and 17B. This preferred structure is formed by a combined spherulitic and lamella nucleation of crystalline ice and its subsequent phase separation from the other solution components of acid and chitosan.

In the absence of the delay interval and achieving an overall solution cooling rate near or greater than about 0.5° C./min, there is a predominance of lamella structure. Generally it is possible to cause predominant lamella nucleation of ice crystals by preferentially cooling one side of a mold containing a warm aqueous solution such that, with time, all of the solution in the mold is cooled. As the ice crystals form and separate from the solution, individual lamella or sheets of ice grow upward into the cooling solution. On removal of the ice by freeze-drying, the lamella type of nucleation provides for open phase separated structures. Lamella type structures have desirable characteristics, e.g., they are highly permeable; they are easily freeze-dried for rapid removal of ice; they have a relatively large pore size (>20 micron) between lamella; and they can be flexible, depending on lamella orientation. However, lamella type structures are often formed of weakly bound regions that are prone to cracking; lamella type structures can be stiff, depending on lamella orientation; and the specific surface area of lamella type structure can be relatively low.

It has been observed that the resting temperature and time of the delay interval allows for promotion of spherulitically nucleated structure within the lamella structure. Spherulitically nucleated structure both complements and modifies the normal lamella chitosan sponge structure. Spherulitic nucleation of ice is generally caused by uniformly cooling an aqueous solution to below its freezing point so that there is a uniform burst of ice crystals throughout the solution. The advantages of spherulitically nucleation type structures, once freeze dried, include (i) they are highly uniform; (ii) they can have a large specific surface area; (iii) they resist cracking; and (iv) they have uniform strength. The inclusion of the delay interval and the hybrid lamella and spherulitically nucleation type structures that result, provide, after freeze drying, a matrix having improved crack resistance and dressing strength uniformity (i.e., suppleness), while retaining sponge permeability.

As shown in FIG. 18, the delayed freezing regime 40 implemented by the controller 34 includes lowering the chitosan biomaterial solution temperature from room temperature to a final temperature below the freezing point, and includes at least one intermediate delay interval 42 that holds a temperature condition for a prescribed period of time at a prescribed increment above the freezing point.

In the illustrated embodiment, the freezing regime 40 includes a first interval 44 that maintains a desired start temperature at or near room temperature (e.g., 20° C.) for a prescribed period of time (e.g., 10 minutes). This assures that the chitosan biomaterial solutions present in all the molds 22 begin the freezing regime 40 at generally the same equilibrium condition.

The freezing regime 40 next drops the temperature to the intermediate temperature, which is held during the delay interval 42. The intermediate temperature is desirably between 2° C. and 10° C. The delay interval 42 is desirably between 20 minutes and 40 minutes. In a representative embodiment, the intermediate temperature is 5° C. and the delay interval 42 is 30 minutes.

It is believed that the delay interval 42 moderates the magnitude of the thermal gradient at the outset of phase separation, as nucleation begins and the spherulites form in the solution. The prescribed intermediate temperature and the duration of delay interval 42 result, at least for a portion of the delay interval 42, in a thermal gradient that approaches zero. In the presence of a low thermal gradient, it is believed that nucleation occurs more uniformly through the volume of chitosan biomaterial solution, allowing adjacent spherulites to form and connect and then open as lamella form, before the chitosan biomaterial solution is exposed to rapid freezing.

The freezing regime 40 includes a final interval 46 that lowers the temperature from the intermediate temperature to the desired final temperature, which is maintained for a prescribed period. In a representative embodiment, the final temperature is minus 40° C., and the prescribed period of time is 50 minutes.

During between each interval 44, 42, and 46 of freezing regime 40, the temperatures may be lowered over a predetermined time period. For example, the freezing temperature of a chitosan biomaterial solution may be lowered from room temperature to the intermediate temperature, or from the intermediate temperature to the final temperature by plate cooling application of a constant temperature cooling ramp of between about −0.4° C./mm to about 0.8° C./mm.

4. Freeze Drying the Chitosan/Ice Matrix

The frozen chitosan/ice matrix desirably undergoes water removal (drying) from within the interstices of the frozen material. This water removal or drying step may he achieved without damaging the structural integrity of the frozen chitosan biomaterial. This may be achieved without producing a liquid phase, which can disrupt the structural arrangement of the ultimate chitosan matrix 12 and 12′. Thus, the ice in the frozen chitosan biomaterial passes from a solid frozen phase into a gas phase (sublimation) without the formation of an intermediate liquid phase. The sublimated gas is trapped as ice in an evacuated condenser chamber at substantially lower temperature than the frozen chitosan biomaterial. Since the spherulitically nucleated structures that are desirably present within the matrix 12 and 12′ often retain considerable moisture due to an impermeable shell structure that forms around the ice core, conditions must be maintained during the water removal step to keep the matrix temperature below its collapse temperature, i.e., the temperature at which the ice core within the structure could melt before it is sublimated.

The preferred manner of implementing the water removal step or drying is by freeze-drying, or lyophilization within the freezer 32. Freeze-drying of the frozen chitosan biomaterial can be conducted by further cooling the frozen chitosan biomaterial. Typically, a vacuum is then applied. Next, the evacuated frozen chitosan material is subject to ramped heating and/or cooling phases in the continued presence of a vacuum.

In a representative embodiment, following the freezing regime 40, freeze drying conditions are maintained for removing water without collapse of the matrix 12 and 12′. In a representative embodiment, for example, a prescribed freeze drying temperature, e.g., minus 50° C. is maintained for a preferred time period (e.g., between 1 and 3 hours), while a vacuum, e.g., in the amount of about 170 mTorr, is applied during this time.

Further freeze drying at higher temperatures may be conducted during subsequent drying phases, while maintaining vacuum pressure. The times and temperatures of the drying phase can change depending upon fill volume, mold configuration, lyophilizer capabilities, etc. Step changes are made to keep the matrix temperature below its collapse temperature. The temperature of the matrix 12 and 12′ is kept as high as possible during the drying phases, but still below the collapse temperature, to provide the shortest cycle time possible. The shelf temperature is ramped up and then down again because high rates of initial sublimation cools the matrix temperature, and as sublimation wanes, matrix temperature increases.

In a representative subsequent primary drying phase, the temperature is (i) ramped over a period of 80 minutes to 30° C., which is then held for 110 minutes; (ii) then lowered over a period of 25 minutes to 14° C., (iii) then further lowered over a period of 180 minutes to minus 6° C., and (iv) then further lowered over a period of 180 minutes to minus 9° C., which is held for a period of 420 minutes. In a representative embodiment subsequent secondary drying phase, the temperature (i) ramped over a period of 120 minutes to 33° C., which is then held for 780 minutes; and (ii) and then lowered back to room temperature (20° C.) and held for a period of 30 minutes.

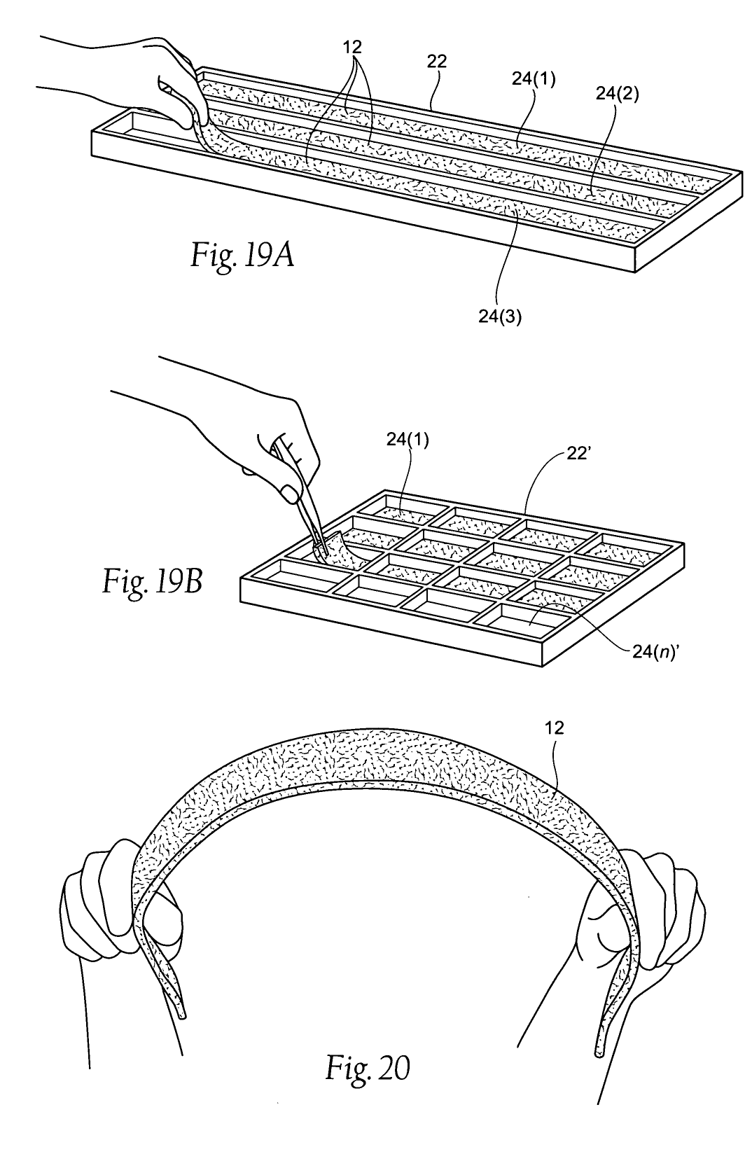

As shown in FIGS. 19A and 19B, the formed, freeze dried matrix 12 and 12′ can be removed from the mold chamber 24(1), 24 (2), and 24(3) and 24(1)′ to 24(n)′. When removed from the mold chamber 24(1), 24(2), and 24 (3) (see FIG. 19A), the formed, freeze-dried matrix 12 measures 28 inches by 2.75 inches, with a thickness of about 0.23 to 0.28 inches. When removed from the mold (see FIG. 20), the formed matrix 12 exhibits inherently suppleness, i.e., it possesses the inherent flexibility and lack of brittleness and stiffness as described above. When removed from the mold chambers 24 (1)′ to 24(n) (see FIG. 19B), the formed freeze-dried matrix 12′ also posseses the same inherent suppleness, as shown in FIG. 4.

When removed from the mold chamber, the dry chitosan matrix 12 and 12′ has a density at or near about 0.03 g/cm³ as a result of the freezing regime 40. For purposes of description, this structure will be called an “uncompressed chitosan matrix.”

D. Subsequent Processing of the Chitosan Matrix

If desired, either dry matrix 12 and 12′ can be subject to further processing to impart other physical characteristics and otherwise optimize the matrix 12 and 12′ for its intended end use.

For low bleeding hemostasis and/or targeted antibacterial/antiviral wound dressing situations, and/or for dental indications, further processing may not be warranted, because the supple uncompressed matrix 12′ (shown ready for use in FIGS. 3 and 4) has, after freezing and freeze-drying as described above, the requisite adhesion strength, cohesion strength, dissolution resistance, flexure, and conformity to perform well in such environments. The uncompressed dry matrix 12′ can be removed from the mold, pouched, and sterilized (as will be described later) without subsequent matrix processing steps. In an alternative arrangement, plastic mold trays can be sized and configured so that, after accommodating freezing and freeze-drying, each mold tray and the dry uncompressed matrix or matrixes it carries can be packaged as an integrated unit, thereby obviating removal of the dry matrix from the mold tray during packaging. In this arrangement, the resulting plastic mold form package provides not only an aesthetic appearance, but also protects the dry matrix against product crushing during handling up to the instant of use.

However, subsequent processing of the matrix may be warranted after drying and prior to packing and sterilization, for example, when the tissue dressing assembly 10 is intended to be, in use, exposed to higher volume blood flow or diffuse bleeding situations, or when exposure to relatively high volume of fluids is otherwise anticipated, as shown in FIG. 5. Representative subsequent matrix processing steps will now be described after freezing and freeze-drying, to provide an assembly 10 of the type shown in FIGS. 1 and 2. However, it should be appreciated that the matrix 12′ of the type shown in FIG. 3 can be subject to one or more or all of these of these subsequent processing steps, if desired.

1. Densification of the Chitosan Matrix

In the illustrated embodiment, the uncompressed dry supple chitosan matrix 12 (FIG. 20) is desirably subject to a densification process. The densification process increases the density of the uncompressed dry chitosan matrix to a threshold density greater than or equal to 0.1 g/cm³, desirably between 0.1 g/cm³ and about 0.5 g/cm³, and most desirably about 0.2 g/cm³. It has been observed that a chitosan matrix at or greater than the threshold density of about 0.1 g/cm³ does not readily dissolve in flowing blood at 37° C.

Following the densification step, the chitosan matrix 12 can be characterized as a supple dry, densified chitosan matrix. It has been observed that the densification process imparts to the densified chitosan matrix 12 significantly increased adhesion strength, cohesion strength and dissolution resistance in the present of blood and liquids.

The physical attributes of the densified dry chitosan matrix 12, in terms of the desired degree of suppleness and desired resistance to dissolution in flowing blood, can be expressed in terms of a ratio between the Gurley stiffness value of the dry matrix (expressed in units of milligrams) (as derived in the manner discussed above) and the density of the dry matrix (expressed in units of g/cm³), which will in shorthand called the dry suppleness-to-density ratio. Desirably, the densified chitosan matrix has a dry suppleness-to-density ratio value of not greater than about 50,000. It is believed that a densified chitosan matrix having a dry suppleness-to-density ratio value of greater than about 50,000 either lacks the requisite resistance to dissolution in flowing blood, or the suppleness or multi-dimensional flexibility to be flexed, bent, folded, twisted, and even rolled upon itself before and during use, without creasing, cracking, fracturing, otherwise compromising the integrity and mechanical and/or therapeutic characteristics of the matrix 12, or a combination of both. Desirably, the densified chitosan matrix has a dry suppleness-to-density ratio value of not greater than about 20,000, and most desirably not greater than about 10,000. A desirable representative range of dry suppleness-to-density ratio values is between about 4000 to about 20,000, and most desirably between about 2000 and about 10,000.

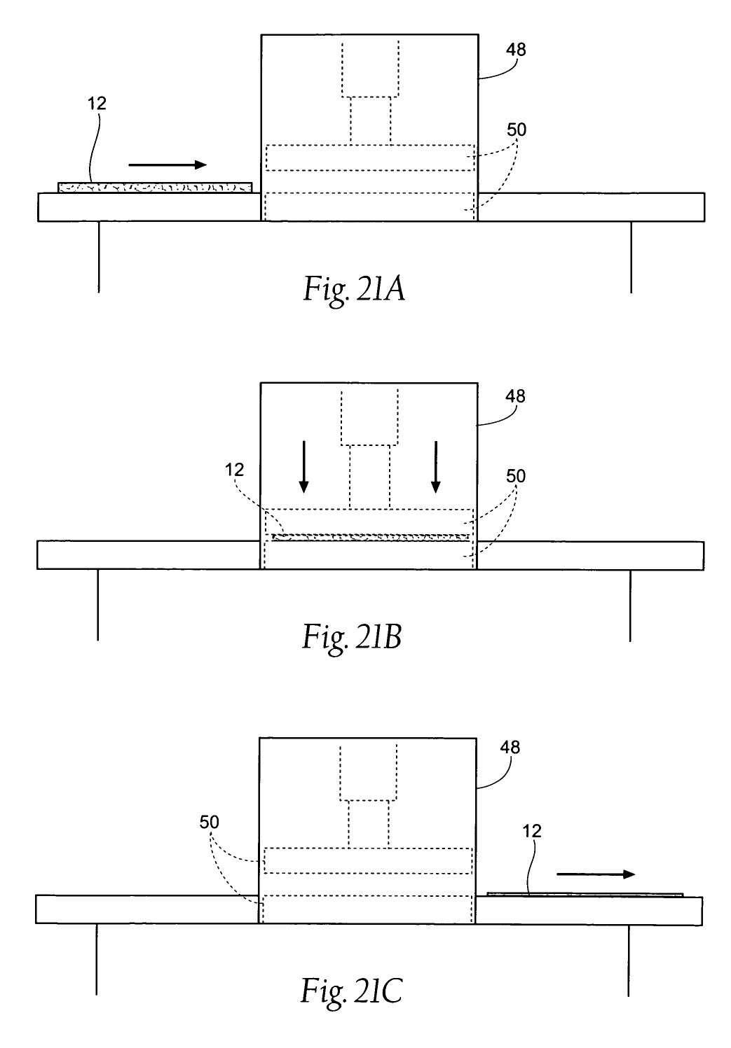

The densification step can be accomplished in various ways. In a representative embodiment (see FIGS. 21A, 21B, and 21C), the uncompressed dry chitosan matrix (FIG. 21A) is placed inside a compression device 48. Inside the device 48, the uncompressed chitosan matrix 12 is compression loaded between heated platens 50 (FIG. 21B). The compression temperature is preferably not less than about 60° C., more preferably it is not less than about 75° C. and not more than about 85° C.

The compression load of the heated platens 50 reduces the thickness of the uncompressed dry matrix 12 from about 0.23 to 0.28 inches to about 0.036 inch (i.e., about 0.9 mm). The compression load thereby increases the density of the uncompressed matrix from about 0.03 g/cm³ to the target density of about 0.2 g/cm³. The supple dry densified chitosan matrix 12 (FIG. 21C) is formed, as is also shown in FIG. 1.

2. Preconditioning of the Densified Supple Chitosan Matrix

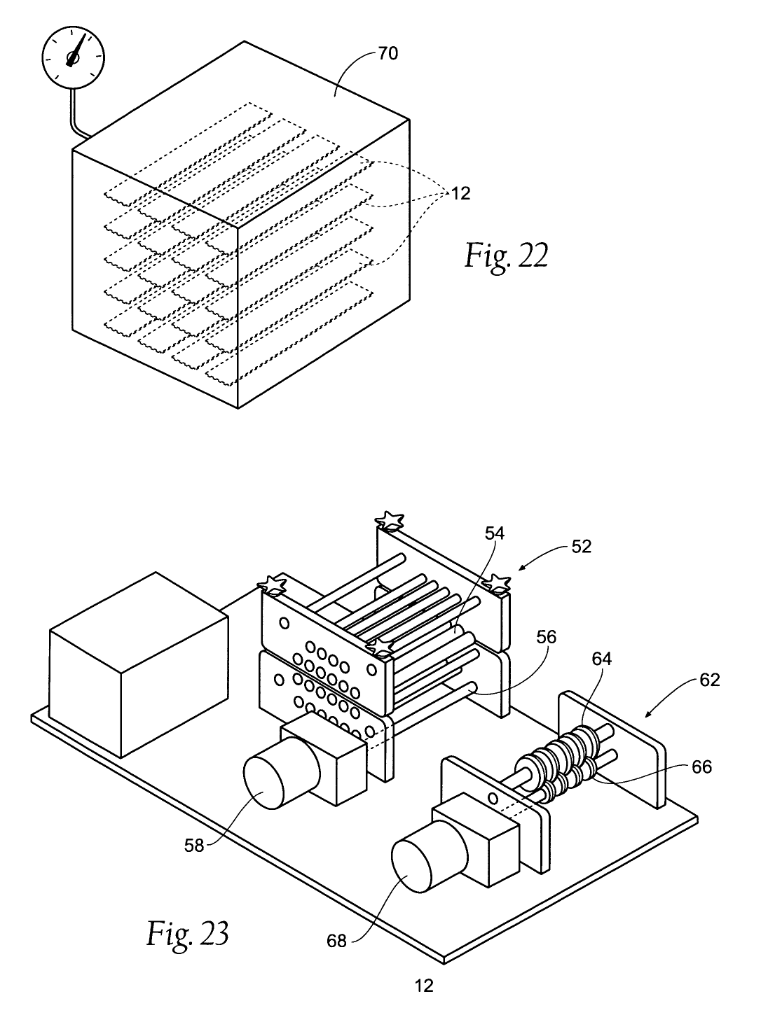

The dry chitosan matrix—now densified—is next preferably preconditioned by heating the densified supple chitosan matrix in an oven 70 (see FIG. 22). The oven 70 can be operated at a temperature of preferably up to about 75° C., more preferably to a temperature of up to about 80° C., and most preferably to a temperature of preferably up to about 85° C. Preconditioning is typically conducted for a period of time up to about 0.25 hours, preferably up to about 0.35 hours, more preferably up to about 0.45 hours, and most preferably up to about 0.50 hours. This pre-conditioning step provides further significant improvement in dissolution resistance with a small cost in a 20-30% loss of adhesion properties.

3. Softening of the Densified Chitosan Matrix

Oven preconditioning as described above can stiffen the supple densified chitosan matrix 12 (raising its Gurley stiffness value). Desirably, after oven conditioning, the supple densified chitosan matrix 12 is subjected to a softening process, which returns inherent suppleness to the matrix and/or lends enhanced flexibility and compliance.

After oven preconditioning and subsequent softening, the dry densified matrix 12 desirably has a Gurley stiffness value (in units of milligrams) (derived as previously discussed) of not greater than about 5000, preferably not greater than about 2500, and most desirably, at or about 1000. Also, after oven preconditioning and subsequent softening, the densified chitosan matrix has a dry suppleness-to-density ratio value of not greater than about 50,000, preferably not greater than about 20,000, and most desirably not greater than about 10,000. A desirable range of dry suppleness-to-density ratio values is between about 4000 to about 20,000, and most desirably between about 2000 and about 10,000.

The softening process can be accomplished by the use of certain plasticizing agents in solution with the chitosan. However, plasticizing may be problematic, because certain plasticizers can change other structural attributes of the assembly 10.

For this reason, the softening process is desirably accomplished by the mechanical manipulation of the supple dry densified chitosan matrix. The mechanical manipulation can be accomplished in various ways. In a representative embodiment (see FIG. 23) the supple dry densified chitosan matrix is passed through a softening device 52.

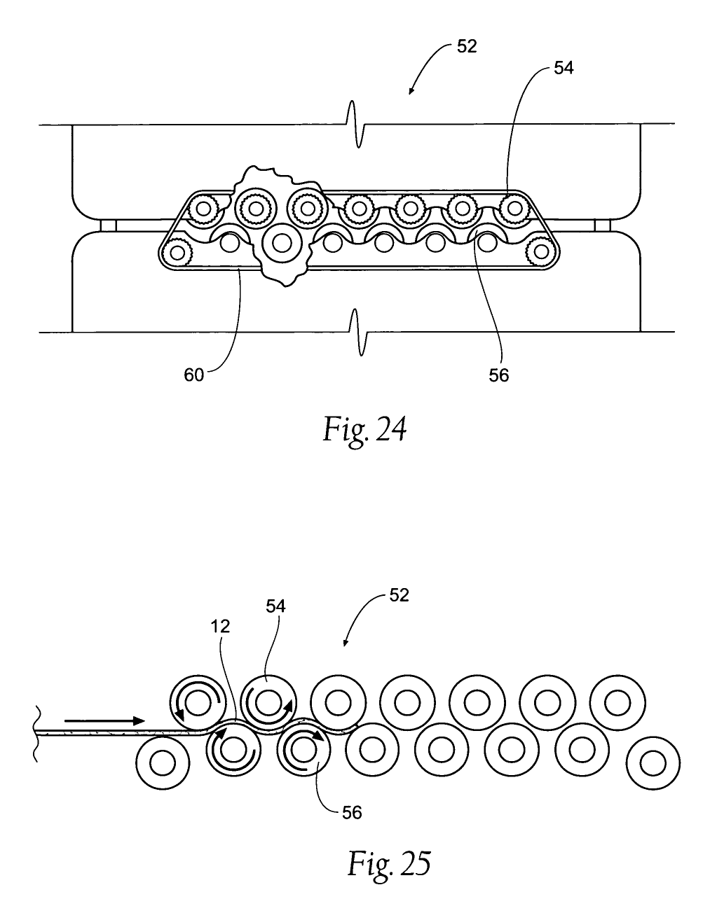

In the illustrated embodiment (see FIG. 24), the softening device 52 comprises an array of upper and lower rollers 54 and 56. The upper rollers 54 are longitudinally spaced apart along parallel axes. The lower rollers 56 are also spaced apart along parallel axes, which are also parallel to the axes of the upper rollers 54. The lower rollers 56 are further arranged in a staggered relationship relative to the upper rollers 54, such that each lower roller 56 is spaced below and between two spaced apart upper rollers 54 (see FIG. 25), providing an undulating path between the upper and lower rollers 54 and 56. The distance between opposing upper and lower rollers 54 and 56 forming the path is slightly less than the thickness of supple densified chitosan matrix.

As a result (see FIG. 25), during passage through the undulating path, the supple densified chitosan matrix 12 is subject to compression or kneading as well as bending along its length axis on both sides of the matrix 12.

A drive motor 58 (see FIG. 23) is linked by a suitable drive mechanism 60 to the rotate the rollers 54 and 56 (see FIG. 25) to draw the supple densified chitosan matrix 12 through one end of the path and discharge the supple densified chitosan matrix 12 from the opposite end of the path.

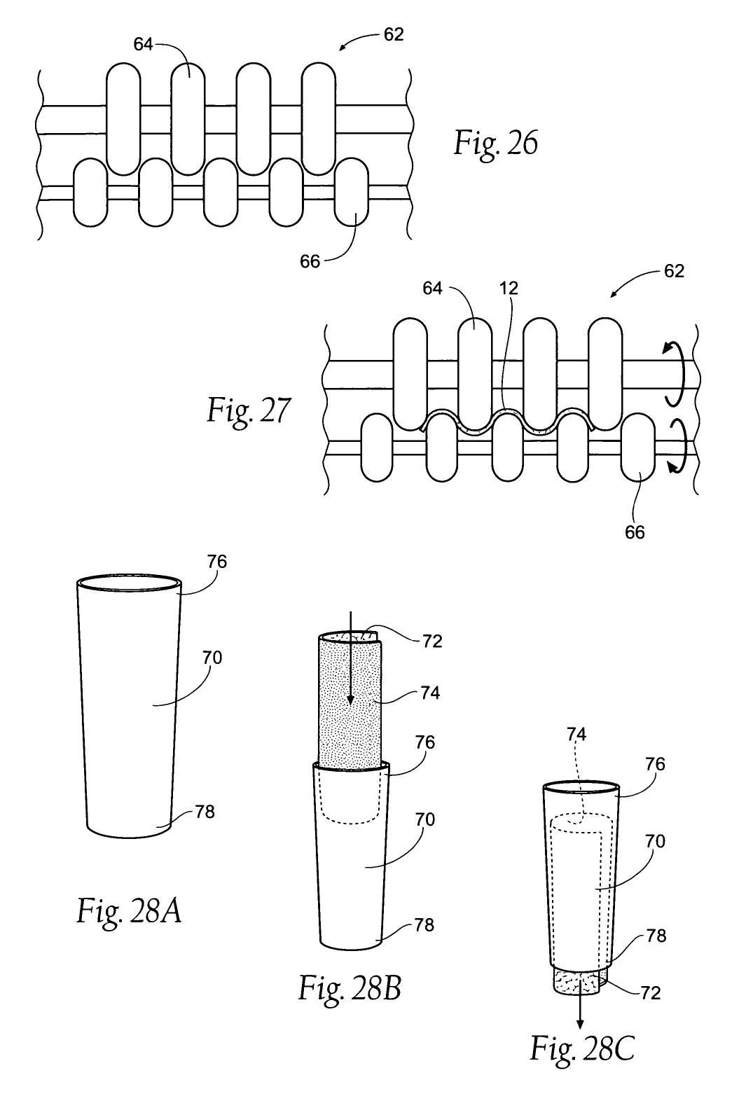

As shown in FIG. 23, the softening device 52, if desired, can further include a second softening array 62, arranged either before or after the first array of upper and lower rollers 54 and 56. The second softening array 62 is sized and arranged to compress or knead the supple densified chitosan matrix 12 along its witdth axis, i.e., along the width of the matrix 12 in a direction ninety degrees from the length compression or kneading provided by the first array of upper and lower rollers 54 and 56. In this arrangement (see FIGS. 26 and 27), the second softening array 62 can comprise an upper and lower array of wheels 64 and 66 arranged for rotation about an axis across the width of matrix 12. The upper wheels 64 are spaced apart along the upper axis, and the lower wheels 66 are spaced apart along the lower axis below and between the spaced apart upper wheels 64 (see FIG. 26). The distance between two upper wheels 64 and an intermediate lower wheel 66 is slightly less than the thickness of supple densified chitosan matrix. A drive motor 68 and suitable drive linkage can be provided to draw the supple densified chitosan matrix 12 between the wheels 64 and 66 (see FIG. 27). During passage between the wheels 64 and 66, the supple densified chitosan matrix is subject to compression or kneading along its longitudinal axis.

In an alternate embodiment (see FIGS. 28A, 28B, and 28C), instead of the second softening array 62 as just described, the matrix 12 can be softened in the width direction after it has been softened by the array of upper and lower rollers 54 and 56 by drawing the matrix width-wise through a tubular tool 70. The tubular tool 70 has a maximum interior diameter that is smaller than the width of the matrix 12. In a representative embodiment (shown in FIG. 28A), for a matrix that is 3 inches wide, the maximum diameter of the tool 70 is 1 inch (circumference 3.14 inches). The matrix 12 is first drawn through the tube with the width curled up towards the crust side 72 (the side facing out of the mold) (FIG. 28B), then drawn again with the width curled up towards the mold side 74 (the side facing the base of the mold) (FIG. 28C). To aid in feeding (as FIG. 28A best shows), the tubular tool 70 preferably has a wider diameter feed neck 76 than exit hole 78, taking the shape of a funnel. Processing through the tool 70 in the manner just described substantially increases flexibility in the width direction, without decrease in in vitro efficacy.

The softening device 52 provides gentle, systematic mechanical softening of the supple densified chitosan matrix 12. The gentle, systematic mechanical softening of the supple densified chitosan matrix improves its inherent suppleness and compliance, without engendering gross failure of the assembly 10 at its time of use.

The softening device 52 as just described can be used to improve the flexibility and compliance of any hydrophilic polymer sponge structure after manufacture, without loss of beneficial features of robustness and longevity of resistance to dissolution. While the methodologies are described in the context of the supple densified chitosan matrix, it should be appreciated that the methodologies are broadly applicable for use with any form of hydrophilic polymer sponge structure, of which the supple densified chitosan matrix 12 is but one example.

The densified, preconditioned, and softened chitosan matrix 12 exhibits all of the above-described characteristics deemed to be desirable for the dressing assembly 10. It also possesses the structural and mechanical benefits that lend robustness and longevity to the matrix during use.

The densified, preconditioned, and softened chitosan matrix 12 makes it possible to readily bend and/or mold the assembly 10 prior to and during placement in or on a targeted injury site. The ability to bend and shape the assembly 10 is especially important when attempting to control strong or deep bleeding. Generally, such bleeding vessels are deep within irregularly shaped wounds. Apposition of the assembly 10 immediately against an injured vessel, and the ability to aggressively stuff the assembly into the wound, is beneficial in the control of such severe bleeding. Furthermore, the more supple and compliant the assembly 10 is, the more resistant it is to tearing and fragmentation as the assembly 10 is made to conform to the shape of the wound and achieve apposition of the assembly 10 with the underlying irregular surface of the injury. Resistance to tearing and fragmentation is a benefit, as it maintains wound sealing and hemostatic efficacy. Compliance and flexibility provide an ability to load a chitosan matrix 12 (e.g., the assembly 10) against a deep or crevice shaped wound without cracking or significant dissolution of the assembly 10.

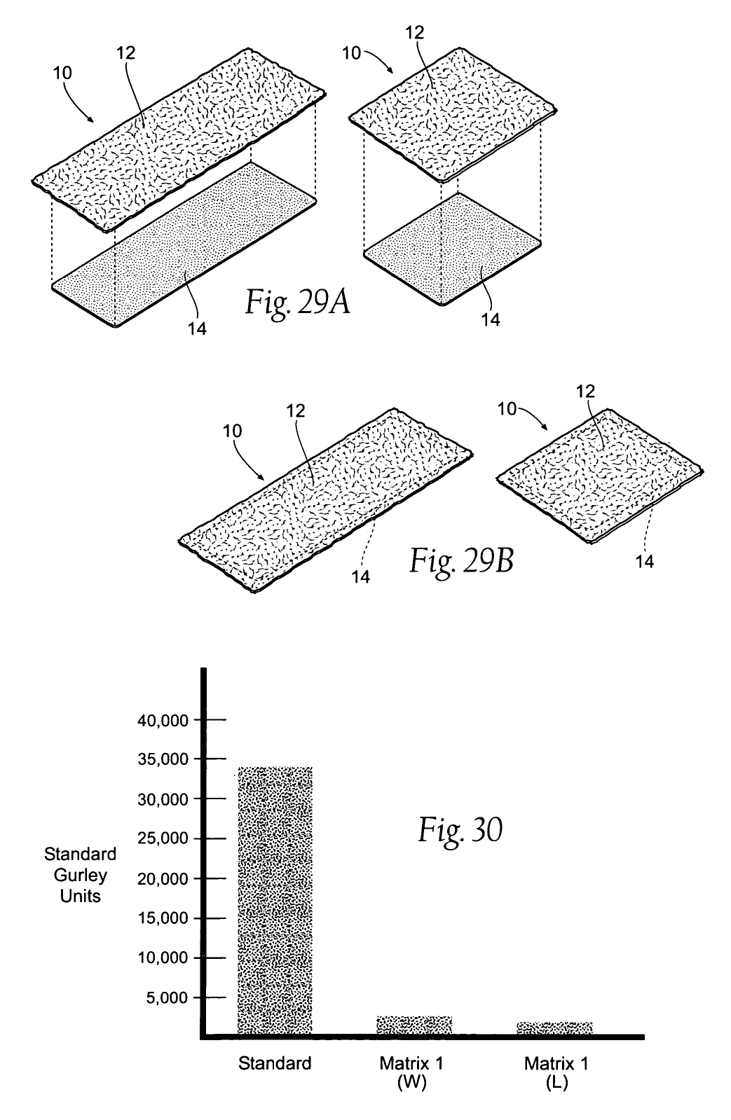

For certain indications, as shown in FIGS. 29A and 29B, it may be desirable to mold the chitosan matrix 12 in the manner described, but in the smaller dressing sizes of the matrix 12′, e.g., 4 inch by 4 inch, or 2 inch by 2 inch, or 2 inch by 4 inch, e.g., by using smaller mold cavities. Alternatively, the smaller dressing sizes can be created after molding by cutting an elongated molded matrix (as shown in FIG. 1) into smaller shaped pieces, either with or without densification. The unique supple sponge structure of the dry chitosan matrix (shown in FIGS. 17A and 17B) makes it possible to readily cut the dry chitosan matrix by conventional cutting means (scissors, knives, saws, or paper cutters) into virtually any desired shape or size, without fracturing or splintering the matrix along the cut lines. The matrix can be cut when in an uncompressed form, with or without subsequent densification, or after densification, and/or preconditioning, and/or softening.

In these smaller sizes, depending upon the particular environment of intended use, it may be desirable, after densification, softening, and preconditioning, but before pouching and sterilization, to apply a backing 14 to the dry chitosan matrix 12, as shown in FIG. 29A. The backing 14 can, if desired, also be applied to an uncompressed chitosan matrix of the type shown in FIG. 3 prior to pouching and sterilization.

The backing 14 can be attached or bonded by direct adhesion to a top or crust layer of a chitosan matrix 12 or 12′ (i.e., the layer that faces out of the mold). Alternatively, an adhesive such as 3M 9942 Acrylate Skin Adhesive, or fibrin glue, or cyanoacrylate glue can he employed. The backing 14 isolates a caregiver's fingers and hand from the fluid-reactive chitosan matrix 12.

E. Placement in the Pouch

The tissue dressing assembly 10 and 10′ can he subsequently packaged in the pouch 16, as previously described, either in a rolled condition or a flattened condition (with or without a backing). The pouch 16 is desirably purged with an inert gas such as either argon or nitrogen gas, evacuated and heat sealed. The pouch 16 acts to maintain interior contents sterility over an extend time (at least 24 months) and also provides a very high barrier to moisture and atmospheric gas infiltration over the same period.

F. Sterilization

After pouching, the tissue dressing assembly 10 and 10′ is desirably subjected to a sterilization step. The tissue dressing assembly 10 can be sterilized by a number of methods. For example, a preferred method is by irradiation, such as by gamma irradiation, which can further enhance the blood dissolution resistance, the tensile properties and the adhesion properties of the wound dressing. The irradiation can be conducted at a level of at least about 5 kGy, more preferably a least about 10 kGy, and most preferably at least about 15 kGy.

### III. PHYSICAL AND CLINICAL CHARACTERISTICS OF THE SUPPLE DRESSING ASSEMBLY

### EXAMPLE 1

### Tensile Strength—Before Sterilization

A dry supple dressing assembly (Matrix 1: 455 g weight of chitosan solution placed in the mold prior to freezing and freeze drying/having a 0.9 mm thickness after densification) was manufactured from a chitosan solution in the manner previously described—i.e. it was frozen according to a freezing regime that placed the chitosan solution at room temperature into a mold, placed the mold on a room temperature shelf, and then brought the shelf to −40° C. in a temperature transition that included a delay interval of 5° C. for 30 minutes, and then subsequently freeze-dried to remove water without collapse of the matrix, and then subsequently densified, preconditioned, and softened, as described above.

Another dry dressing assembly (Matrix 2: 455 g weight of chitosan solution placed in the mold prior to freezing and freeze drying/and having a 0.9 mm thickness after densification) was manufactured from the same chitosan solution using a freezing regime that placed the chitosan solution at room temperature into a mold that was placed on −40° C. shelf without an intermediate delay interval, and then subsequently freeze dried to remove water without collapse of the matrix, and then subsequently densified and preconditioned (without softening).

Neither Matrix 1 nor Matrix 2 were subjected to gamma sterilization prior to testing.

Dry samples of Matrix 1 (n=18) and Matrix 2 (n=18) were subjected to tensile strength testing using an Instron™ device (ASTM Method D412 (Method A, Section 12). Samples were taken from both ends of the matrix (OR & IR) as well as from the middle region (MR). Three samples from each region were tested for horizontal tensile strength and vertical tensile strength. The vertical direction is tensile strength (expressed in Newtons) oriented along the width of the matrix sample, while the horizontal direction is tensile strength (expressed in Newtons) along the length of the matrix sample. The crosshead speed was 50 mm/min. Each test piece was a bar 1.27 cm wide (0.5″) and 6.99 cm long (2.75″). Duct tape was placed on the top and bottom 1.9 cm (0.75″) to avoid damaging the test piece ends when gripping and to ensure failure wass always in the middle test region (3.18 cm or 1.25″).

The following Table summarizes the results of the testing:

TABLE 1
MAXIMUM LOAD TENSILE STRENGTHS BEFORE
STERILIZATION

| | MATRIX 2 | | MATRIX 1 | |
|---|---|---|---|---|
| | 455 g/0.9 mm | | 455 g/0.9 mm | |
| Position | Vertical | Horizontal | Vertical | Horizontal |
| OR | 3.70366 | 8.56534 | 7.06565 | 20.78348 |
| | 5.83563 | 13.68812 | 8.22033 | 10.31058 |
| | 5.63845 | 8.25216 | 10.44571 | 26.95859 |
| MR | 7.02448 | 14.20763 | 17.96917 | 31.39515. |
| | 5.52157 | 10.37916 | 16.34544 | 24.15838 |
| | 4.75477 | 7.7611 | 18.47541 | 27.96895 |
| IR | 3.5381 | 16.75244 | 21.4069 | 15.29328 |
| | 5.79096 | 24.1702 | 18.33673 | 12.98342 |
| | 15.3386 | 9.2226 | 19.83852 | 25.5036 |
| Ave | 6.3 | 12.6 | 15.3 | 21.7 |
| Stdev | 3.5 (55.8%) | 5.4 (42.7%) | 5.3 (34.7%) | 7.3 (33.7%) |

The test results demonstrate that, although the thickness and density for the dry Matrix 1 and dry Matrix 2 are the same, the tensile orientations strengths of Matrix 1 and Matrix 2 before sterilization are very different. The dry Matrix 1 and dry Matrix 2 tensile strengths (before sterilization) are, respectively 21.7 and 12.6 (Horizontal) and 15.3 and 6.3 (Vertical). The test results demonstrate a significant tensile advantage (both horizontally and vertically) in Matrix 1. Further, the coefficient of variation in tensile strength is near 50% for Matrix 2 while it is nearer 30% for Matrix 1, indicating enhanced uniformity in the Matrix 1.

### EXAMPLE 2

### Tensile Strength—after Gamma Sterilization

Dry samples of Matrix 1 (n=18) and Matrix 2 (n=18) (from the same lot as described in Example 1) were subjected to tensile strength testing after undergoing sterilization by gamma irradiation at 15 kGy. The Instron™ device was used for testing the samples, and ASTM Method D412 (Method A, Section 12) was observed. After gamma sterilization, dry samples were taken from both ends of the matrix (OR & IR) as well as from the middle region (MR). As in Example 1, three dry samples from each region were tested for horizontal tensile strength and vertical tensile strength. The vertical direction is tensile strength (Newtons) oriented along the width of the matrix sample, while the horizontal direction is tensile strength (Newtons) along the length of the matrix sample.

The following Table summarizes the results of the testing:

TABLE 2
MAXIMUM LOAD TENSILE STRENGTHS AFTER GAMMA
STERILIZATION

| | MATRIX 2 | | MATRIX 1 | |
|---|---|---|---|---|
| | 455 g/0.9 mm | | 455 g/0.9 mm | |
| Position | Vertical | Horizontal | Vertical | Horizontal |
| OR | 3.82788 | 9.10287 | 10.38337 | 16.11729 |
| | 5.20685 | 4.65672 | 8.99664 | 9.1101 |
| | 3.98338 | 5.64517 | 11.0916 | 13.5463 |
| MR | 3.95973 | 11.00548 | 10.34841 | 17.3211 |
| | 3.21287 | 11.15282 | 8.34735 | 7.10421 |
| | 4.16728 | 6.90572 | 13.10092 | 13.12256 |
| IR | 5.48557 | 7.89183 | 22.31267 | 13.98774 |
| | 9.78998 | 7.71209 | 8.45192 | 6.67215 |
| | 9.19211 | 8.54624 | 18.56407 | 13.41097 |
| Ave | 5.4 | 8.1 | 12.4 | 12.3 |
| Stdev | 2.4 (44.5%) | 2.2 (27.2%) | 4.9 (39.3%) | 3.8 (30.9%) |

Like Example 1, the test results of Example 2 demonstrate that, although the thickness and density for the dry Matrix 1 and dry Matrix 2 are the same, the tensile orientations strengths of dry Matrix 1 and dry Matrix 2 after sterilization are also very different. The Matrix 1 and Matrix 2 tensile strengths (after sterilization) are, respectively 12.3 and 8.1 (Horizontal) and 12.4 and 5.4 (Vertical). The test results demonstrate a significant tensile advantage (both horizontally and vertically) (after sterilization) in Matrix 1. Further, the coefficient of variation in tensile strength for Matrix 1 remains near 30% both horizontally and vertically, which, like Example 1, demonstrates the remarkable uniformity of the construct.

### EXAMPLE 3

### In Vivo Animal Testing

Tissue dressing assemblies comprising Matrix 1, as described in Example 1, were applied to abdominal aorta 4 mm diameter perforation injuries in an animal model (swine). A total of sixteen tissue dressing assemblies were applied to eight different animals, two to each animal, one mold side up and the other mold side down. Success was indicated if hemostasis was achieved for more than 30 minutes.

Fourteen (14) of sixteen (16) tissue dressing assemblies achieved success.

In addition, a tissue dressing assembly comprising Matrix 1 was tested in a through and through wound in the animal model, in which the femoral artery and vein were severed. The tissue dressing assembly was found to be readily stuffable into the wound and maintained hemostasis for over three hours, until the animal was sacrificed.

### EXAMPLE 4

### Burst Strengths

The adhesive characteristics of a tissue dressing assembly comprising a Matrix 1 (as above described) were tested and verified using a test fixture specially designed for the task, as described in copending U.S. patent application Ser. No. 11/020,365, filed Dec. 23, 2004, which is incorporated herein by reference. The test fixture provides a platform that simulates an arterial wound sealing environment. The test fixture makes it possible to assess, for that environment and exposure period, the burst (or rupture) strength of a given hydrophilic polymer sponge structure, or a manufactured lot of such structures, in a reproducible and statistically valid way that statistically correlates with in vivo use. The highest pressure state (burst strength, expressed in mmHg) observed is compared to a prescribed “pass-fail” criteria. In a representative example, burst strengths greater than 750 mmHg indicate a “pass.” Burst strengths below 750 mmHg indicate a “fail.” This criteria imposes a strict “pass” standard, as it represents a pressure level that is generally six times greater than normal human blood systolic pressure.

Three Groups, each with sixteen tissue dressing assemblies comprising a Matrix 1, were subjected to burst testing using the fixture, with mold side up and mold side down. The results for each Group is summarized below.

Group 1

| | Mold Side | Mold Side | | Diff from |
|---|---|---|---|---|
| | Up | Down | Group of 4 | Avg |
| 1-6b | 878 | 959 | 1045 | 0% |
| 2-4a | 1037 | 920 | | |
| 3-1a | 1153 | 897 | | |
| 3-7a | 1194 | 1320 | | |
| 4-6a | 1049 | 1110 | 1065 | 2% |
| 5-3a | 1008 | 1049 | | |
| 6-6c | 1018 | 1156 | | |
| 6-8b | 1093 | 1035 | | |
| 7-5a | 1158 | 1031 | 1023 | −2% |
| 9-3c | 1169 | 1142 | | |
| 10-2c | 914 | 739 | | |
| 10-8c | 1003 | 1031 | | |
| 11-4b | 1042 | 959 | 1061 | 1% |
| 12-2a | 1188 | 1032 | | |
| 12-8a | 1147 | 1019 | | |
| 13-5a | 1065 | 1037 | | |
| Avg. | 1070 | 1027 | | |
| | | | every other 4 | |
| | 1049 | | 1023 | −2% |
| | 113 | | 1068 | 2% |
| | 11% | | 1005 | −4% |
| | | | 1097 | 5% |

Group 2

| | Mold Side | Mold Side | | Diff from |
|---|---|---|---|---|
| | Up | Down | Group of 4 | Avg |
| 1-2b | 830 | 1069 | 1064 | 2% |
| 1-8b | 1082 | 1007 | | |
| 2-6a | 1061 | 1076 | | |
| 3-3a | 1286 | 1102 | | |
| 4-2a | 977 | 975 | 1002 | −4% |
| 4-8a | 803 | 964 | | |
| 5-5a | 1097 | 983 | | |
| 6-2c | 1230 | 989 | | |
| 7-1a | 992 | 864 | 992 | −5% |
| 7-7a | 1005 | 1039 | | |
| 9-5c | 871 | 1062 | | |
| 10-4c | 956 | 1145 | | |
| 11-6b | 1190 | 1129 | 1104 | 6% |
| 12-4a | 1137 | 1172 | | |
| 13-1 | 1230 | 1073 | | |
| 13-7 | 1038 | 862 | | |
| Avg | 1049 | 1032 | | |
| | | | every other 4 | |
| | | | 1003 | −4% |
| | 1041 | | 1026 | −1% |
| | 119 | | 1057 | 2% |
| | 11% | | 1076 | 3% |

Group 3

| | Mold Side | Mold Side | | Diff from |
|---|---|---|---|---|
| | Up | Down | Group of 4 | Avg |
| 3-5a | 1341 | 1180 | 1259 | 3% |
| 2-8a | 1257 | 1358 | | |
| 4-4a | 1080 | 1264 | | |
| 13-3a | 1417 | 1173 | | |
| 11-2b | 1197 | 976 | 1217 | −1% |
| 10-6b | 1230 | 1188 | | |
| 11-8b | 1429 | 1233 | | |
| 12-6a | 1257 | 1226 | | |
| 2-2a | 1044 | 1269 | 1111 | −9% |
| 1-4b | 1035 | 1078 | | |
| 5-1a | 1131 | 1173 | | |
| 9-1c | 1147 | 1011 | | |
| 5-7a | 1529 | 1207 | 1312 | 7% |
| 9-7c | 1457 | 1253 | | |
| 6-4c | 1132 | 1138 | | |
| 7-3a | 1533 | 1243 | | |
| Avg. | 1264 | 1186 | | |
| | | | every other 4 | |
| | | | 1218 | −1% |
| | 1225 | | 1232 | 1% |
| | 141 | | 1198 | −2% |
| | 12% | | 1251 | 2% |

Within each Group of sixteen dressings tested, the quantity of dressings required to accurately represent the entire load was determined. For each Group, collections of four burst pressure results were averaged and the actual values compared against the average.

Analyzing eight sets of four burst pressure results for each Group (32 sets of 4) resulted in an average deviation from average burst pressure of just 2.2%. The three highest variances were 9%, 7% and 6%. Nineteen sets of 4 dressings had 4% deviation or less.

Examples 1 and 2 demonstrate a coefficient of variation in tensile strength for Matrix 1 that indicates uniformity of structure among lots of Matrix 1 structures. This Example 4 further demonstrates a low standard of deviation of burst strengths among lots of Matrix 1 structures (<10%), further indicating the overall uniformity in structure that is achieved with Matrix 1 structures.

### EXAMPLE 5

### Flexure Testing

The flexural characteristics of a dry tissue dressing assembly comprising a Matrix 1 (as above described) (thickness 0.9 mm) were tested using a Gurley Stiffness Tester Model 4171D manufactured by Gurley Precision Instruments of Troy, N.Y., and Gurley ASTM D6125-97, along the width (w) and length (L) of the matrix. This test method determines the bending resistance of flexible flat-sheet materials by measuring the force required to bend a specimen under controlled conditions. Standard Gurley Units are expressed in units of milligrams. Lower Standard Gurley Unit values indicate lesser resistance to flexure, i.e., greater suppleness.

These flexural characteristics were compared to the flexural characteristics of a commercially available densified chitosan matrix (the HemCon® Bandage, thickness 5.5 mm), which is the current industry standard. The HemCon® Bandage includes a chitosan matrix that is formed by a freezing, lyophilization, densification and pretreatment process, but does not includes a delay interval in the freezing process or a softening step, as described above.

FIG. 30 shows the results of the flexural testing, expressed in Standard Gurley Units (mean values n=8). The Standard Gurley Values of the Matrix 1 were about 2500 Standard Gurley Units (width) and about 1000 Standard Gurley Units (length). The Standard Gurley Values for the state of the art HemCon® Bandage are about 34,000 (mean tensile strength for the HemCon® Bandage is about 75 Newtons, and its density is about 0.2 g/cm³).

FIG. 30 demonstrates the significantly improved flexibility of a Matrix 1 structure, both along its width (W) and along its length (L), compared to the state of the art HemCon® Bandage.

Based upon the tensile strength data obtained in Example 2 (after sterilization) and the flexural test data obtained in this Example 5, it can be seen that the densified material of dry Matrix 1 possesses a dry suppleness-to-strength ratio of about 208 (width direction) and about 83 (length direction). In contrast, the state of the art HemCon® Bandage possesses a dry suppleness-to-strength ratio value of about 453.

Also based upon the flexural test data obtained in this Example 5, it can be seen that the densified material of dry Matrix 1 (having a density about 0.2 g/cm³) possesses a dry suppleness-to-density ratio value of about 12,500 (width direction) and about 5000 (length direction). In contrast, the state of the art HemCon® Bandage possesses a dry suppleness-to-density ratio value of about 170,000.

### IV. INDICATIONS AND CONFIGURATIONS FOR THE SUPPLE CHITOSAN MATRIX

The foregoing disclosure has focused upon the use of the tissue dressing assembly 10 and 10′ principally in the setting of stanching blood and/or fluid loss at a wound site. Other indications have been mentioned, and certain of these and other additional indications now will be described in greater detail.

Of course, it should be appreciated by now that the remarkable technical features that a supple hydrophilic polymeric sponge structure, of which the chitosan matrix is but one example, possesses can be incorporated into dressing structures of diverse shapes, sizes, and configurations, to serve a diverse number of different indications. As will be shown, the shapes, sizes, and configurations that a given supple sponge structure (e.g., the chitosan matrix 12 and 12′) can take are not limited to the assembly 10 and 10′ described, and can transform according to the demands of a particular indication. Several representative examples follow, which are not intended to be all inclusive or limiting.

A. Body Fluid Loss Control (e.g., Burns)

The control of bleeding represents but one indication where preservation of a body fluid is tantamount to preserving health and perhaps life. Another such indication is in the treatment of burns.

Burns can occur by exposure to heat and fire, radiation, sunlight, electricity, or chemicals. Thin or superficial burns (also called first-degree burns) are red and painful. They swell a little, turn white when you press on them, and the skin over the burn may peel off in one or two days. Thicker burns, called superficial partial-thickness and deep partial-thickness burns (also called second-degree burns), have blisters and are painful. There are also full-thickness burns (also called third-degree burns), which cause damage to all layers of the skin. The burned skin looks white or charred. These burns may cause little or no pain if nerves are damaged.

The presence of a tissue burn region compromises the skin's ability in that region to control fluid loss (leading to dehydration), as well as block entry of bacteria and microbes. Therefore, in the treatment of all burns, dressings are used to cover the burned area. The dressing keeps air off the area, reduces pain and protects blistered skin. The dressing also absorbs fluid as the tissue burn heals. Anti-microbial creams or ointments and/or moisturizers are also used to prevent drying and to ward off infection.

A supple, densified hydrophilic polymer sponge structure (e.g., a chitosan matrix 12 of the type already described) can be used to treat a tissue burn region. The supple, densified hydrophilic polymer sponge structure (e.g., chitosan matrix 12) will absorb fluids and adhere to cover the burn region. The supple, densified hydrophilic polymer sponge structure (e.g., the chitosan matrix 12) can also serve an anti-bacterial/anti-microbial protective barrier at the tissue burn region.

B. Antimicrobial Barriers

In certain indications, the focus of treatment becomes the prevention of ingress of bacteria and/or microbes through a tissue region that has been compromised, either by injury or by the need to establish an access portal to an interior tissue region. Examples of the latter situation include, e.g., the installation of an indwelling catheter to accommodate peritoneal dialysis, or the connection of an external urine or colostomy bag, or to accomplish parenteral nutrition, or to connect a sampling or monitoring device; or after the creation of an incision to access an interior region of the body during, e.g., a tracheotomy, or a laparoscopic or endoscopic procedure, or the introduction of a catheter instrument into a blood vessel.

A supple hydrophilic polymer sponge structure (with or without densification, e.g., a chitosan matrix 12 or 12′ of the type already described) can be readily sized and configured for use as an antimicrobial gasket. The gasket can be sized and configured to be placed over an access site, e.g., an access site where an indwelling catheter and the like resides. The gasket can include a pass-through hole, which allows passage of the indwelling catheter through it. It should be appreciated that, in situations where there is only an incision or access site without a resident catheter, the anti-microbial component will not include the pass-through hole.

C. Treatment of Staph and MRSA Infections

The focus of treatment can also be after exposure to Staphylococcus aureus bacteria (staph) in general and/or to methicillin resistant Staphylococcus aureus (MRSA) in particular. MRSA is a type of Staphylococcus aureus bacteria that is resistant to antibiotics including methicillin, oxacillin, penicillin and amoxicillin. While 25% to 30% of the population is colonized with staph, approximately 1% is colonized with MRSA.

Staph infections, including MRSA, occur most frequently among persons in hospitals and healthcare facilities (such as nursing homes and dialysis centers) who have weakened immune systems. These healthcare-associated staph infections include surgical wound infections, urinary tract infections, bloodstream infections, and pneumonia. Staph and MRSA can also cause illness in persons outside of hospitals and healthcare facilities. MRSA infections that are acquired by persons who have not been recently (within the past year) hospitalized or had a medical procedure (such as dialysis, surgery, catheters) are know as CA-MRSA infections. Staph or MRSA infections in the community are usually manifested as skin infections, such as pimples and boils, and occur in otherwise healthy people.

The main mode of transmission of MRSA is via hands (especially health care workers' hands) which may become contaminated by contact with a) colonized or infected patients, b) colonized or infected body sites of the personnel themselves, or c) devices, items, or environmental surfaces contaminated with body fluids containing MRSA. In addition, recent reports show a link between tattooing and MRSA. Topically, attempts to treat the infections include the use of antimicrobial dressings made with silver or polyhexamethylene biguanide (PHMB). There are problems associated with current wound dressings, such as lack of fluid retention, high risk of maceration due to over-saturation of the wound bed, and inability to maintain an optimally moist wound environment.

A supple hydrophilic polymer sponge structure (with or without densification, e.g., a chitosan matrix 12 or 12′ of the type already described) can be used to treat a site of infection by staph or MRSA. The supple hydrophilic polymer sponge structure (e.g., chitosan matrix 12 or 12′) will absorb fluids and adhere to cover the infection site. The supple hydrophilic polymer sponge structure (e.g., the chitosan matrix 12 or 12′) can also serve an anti-bacterial/anti-microbial protective barrier at the infection site. The excellent adhesive and mechanical properties of the densified supple matrix 12 make it eminently suitable for use in such applications on the extremity (epidermal use) and inside the body. Such applications would include short to medium term (0-120 hour) control of infection and bleeding at catheter lead entry/exit points, at entry/exit points of biomedical devices for sampling and delivering application, and at severe injury sites when patient is in shock and unable to receive definitive surgical assistance.

D. Antiviral Patches

There are recurrent conditions that are caused by viral agents.

For example, herpes simplex virus type 1 (“HSV1”) generally only infects those body tissues that lie above the waistline. It is HSV1 that causes cold sores in the majority of cases. Cold sores (or lesions) are a type of facial sore that are found either on the lips or else on the skin in the area near the mouth. Some equivalent terminology used for cold sores is “fever blisters” and the medical term “recurrent herpes labialis”.

Herpes simplex virus type 2 (“HSV2”) typically only infects those body tissues that lie below the waistline.” It is this virus that is also known as “genital herpes”. Both HSV 2 (as well as HSV1) can produce sores (also called lesions) in and around the vaginal area, on the penis, around the anal opening, and on the buttocks or thighs. Occasionally, sores also appear on other parts of the body where the virus has entered through broken skin.

A supple hydrophilic polymer sponge structure (with or without densification, e.g., a chitosan matrix 12 or 12′ of the type already described) can be used as an anti-viral patch assembly, for placement over a surface lesion of a type associated with HSV1 or HSV2, or other forms of viral skin infections, such as molluscum contagiosum and warts. The excellent adhesive and mechanical properties of the supple, densified matrix 12 make it eminently suitable for use in anti-viral applications on the extremity (epidermal use) and inside the body. The presence of the anti-viral patch formed from the matrix 12 can kill viral agents and promote healing in the lesion region.

E. Bleeding Disorder Intervention

There are various types of bleeding or coagulation disorders. For example, hemophilia is an inherited bleeding, or coagulation, disorder. People with hemophilia lack the ability to stop bleeding because of the low levels, or complete absence, of specific proteins, called “factors,” in their blood that are necessary for clotting. The lack of clotting factor causes people with hemophilia to bleed for longer periods of time than people whose blood factor levels are normal or work properly. Idiopathic thrombocytopenic purpura (ITP) is another blood coagulation disorder characterized by an abnormal decrease in the number of platelets in the blood. A decrease in platelets can result in easy bruising, bleeding gums, and internal bleeding.

A supple, densified matrix (e.g., the chitosan matrix 12) can be sized and configured to be applied as an interventional dressing, to intervene in a bleeding episode experience by a person having hemophilia or another coagulation disorder. As previously described, the presence of the chitosan matrix 12 attracts red blood cell membranes, which fuse to chitosan matrix 12 upon contact. A clot can be formed very quickly and does not need the clotting proteins that are normally required for coagulation. The presence of the chitosan matrix 12 during a bleeding episode of a person having hemophilia or other coagulation disorder can accelerate the clotting process independent of the clotting cascade, which, in such people, is in some way compromised. For this reason, the presence of the chitosan matrix 12 on a dressing can be effective as an interventional tool for persons having a coagulation disorder like hemophilia.

F. Controlled Release of Therapeutic Agents

A supple densified matrix (e.g., the chitosan matrix 12 as previously described) can provide a topically applied platform for the delivery of one or more therapeutic agents into the blood stream in a controlled release fashion. The therapeutic agents can be incorporated into the matrix structure, e.g., either before or after the freezing step, and before the drying and densification steps. The rate at which the therapeutic agents are released from the matrix structure can be controlled by the amount of densification. The more densified the hydrophilic polymer sponge structure is made to be, the slower will be the rate of release of the therapeutic agent incorporated into the structure.

Examples of therapeutic agents that can be incorporated into a hydrophilic polymer sponge structure (e.g., the chitosan matrix 12) include, but are not limited to, drugs or medications, stem cells, antibodies, anti-microbials, anti-virals, collagens, genes, DNA, and other therapeutic agents; hemostatic agents like fibrin; growth factors; and similar compounds.

G. Mucosal Surfaces

The beneficial properties of the supple, densified chitosan matrix 12 includes adherence to mucosal surfaces within the body, such as those lining the esophagus, gastro-intestinal tract, urinary tract, the mouth, nasal passages and airways, and lungs. This feature makes possible the incorporation of the chitosan matrix 12, e.g., in systems and devices directed to treating mucosal surfaces where the adhesive sealing characteristics, and/or accelerated clotting attributes, and/or anti-bacterial/anti-viral features of the chitosan matrix 12, as described, provides advantages. Such systems and methods can include the anastomosis of bowels and other gastro-intestinal surgical procedures, repairs to esophageal or stomach function, sealing about sutures, etc.

H. Dental

There are various dental procedures for intervening when conditions affecting the oral cavity and its anatomic structures arise. These procedures are routinely performed by general practitioners, dentists, oral surgeons, maxillofacial surgeons, and peridontistics.

During and after conventional dental procedures—e.g., endodontic surgery, or periodontal surgery, orthodontic treatment, tooth extractions, orthognathic surgery, biopsies, and other oral surgery procedures—bleeding, fluid seepage or weeping, or other forms of fluid loss typically occur. Bleeding, fluid seepage or weeping, or other forms of fluid loss can also occur in the oral cavity as a result of injury or trauma to tissue and structures in the oral cavity. Swelling and residual bleeding can be typically expected to persist during the healing period following the procedure or injury, while new gum tissue grows.

A supple matrix structure (with or without densification, e.g., the chitosan matrix 12 or 12′ described herein) can be shaped, sized, and configured for placement in association with tissue or bone in an oral cavity or an adjacent anatomic structure. The supple matrix structure can be used in various dental surgical procedures, e.g., a tooth extraction; or endodontic surgery; or periodontal surgery; or orthodontic treatment; or orthognathic surgery; or a biopsy; or gingival surgery; or osseous surgery; or scaling or root planning; or periodontal maintenance; or complete maxillary or mandibular denture; or complete or partial denture adjustment; or denture rebase or reline; or soft tissue surgical extraction; or bony surgical extraction; or installation of an occlusal orthotic device or occlusal guard or occlusal adjustment; or oral surgery involving jaw repair; treatment of cystic cavity defects in the jaw; or new bone growth or bone growth promotion; or any other surgical procedure or intervention affecting tissue in the oral cavity, anatomic structures in the oral cavity, or alveolar (jaw) bone. The supple matrix structure makes it possible to stanch, seal, or stabilize a site of tissue or bone injury, tissue or bone trauma, or tissue or bone surgery. The supple matrix structure can also form an anti-microbial or anti-viral barrier; and/or promote coagulation; and/or release a therapeutic agent; and/or treat a periodontal or bone surface; and/or

combinations thereof.

### V. CONCLUSION

It has been demonstrated that a supple hydrophilic polymer sponge structure, like the densified chitosan matrix 12 or the uncompressed chitosan matrix 12′, can be readily adapted for association with dressings or platforms of various sizes and configurations, such that a person of ordinary skill in the medical and/or surgical arts could adopt any supple hydrophilic polymer sponge structure, like the chitosan matrix 12 or 12′, to diverse indications on, in, or throughout the body.

Therefore, it should be apparent that above-described embodiments of this invention are merely descriptive of its principles and are not to be limited. The scope of this invention instead shall be determined from the scope of the following claims, including their equivalents.

What is claimed is:

1. A supple sponge structure adapted for placement in contact with animal tissue comprising a biocompatible hydrophilic polymer form, the form having been softened by a mechanical softening device comprising a plurality of spaced apart upper and lower rolling surfaces defining between them an undulating path sized and arranged to knead the form along an axis.

2. A supple sponge structure according to claim 1, wherein the form has, after softening, a resistance to flexure expressed as a dry Gurley stiffness value (in units of milligrams) of not greater than about 5000.

3. A supple sponge structure according to claim 1, wherein the form has, after softening, a dry Gurley stiffness value (in units of milligrams) of not greater than about 2000.

4. A supple sponge structure according to claim 3, wherein the form has, after softening, a dry suppleness to strength ratio of not greater than about 210, the dry suppleness-to-strength ratio comprising a ratio between the dry Gurley stiffness value (in units of milligrams) and tensile strength (in units of Newtons).

5. A supple sponge structure according to claim 1, wherein the form has, after softening, a dry Gurley stiffness value (in units of milligrams) of not greater than about 1000.

6. A supple sponge structure according to claim 5, wherein the form has, after softening, a dry suppleness-to strength ratio of not greater than about 210, the dry suppleness-to-strength ratio comprising a ratio between the Gurley stiffness value (in units of milligrams) and tensile strength (in units of Newtons).

7. A supple sponge structure according to claim 1, wherein the form has an initial sponge density that is increased to a densified sponge density by application of mechanical pressure.

8. A supple sponge structure according to claim 7, wherein the densified sponge density comprises about 0.1 g/cm³ to about 0.5 g/cm³.

9. A supple sponge structure according to claim 8, wherein the densified sponge density comprises about 0.2 g/cm³.

10. A supple sponge structure according to claim 7, wherein the form has, after softening, a dry stiffness-to-strength ratio of not greater than about 50,000, the dry suppleness-to-density ratio comprising a ratio between a Gurley stiffness value (in units of milligrams) and the densified sponge density (in units of g/cm³).

11. A supple sponge structure according to claim 10, wherein the dry suppleness-to-density ratio is not greater than about 20,000.

12. A supple sponge structure according to claim 10, wherein the dry suppleness-to-density ratio is not greater than about 10,000.

13. A supple sponge structure according to claim 1, wherein the form, after softening, has a sponge thickness of not greater than about 8 mm.

14. A supple sponge structure according to claim 1, wherein the form has an initial sponge thickness of not greater than about 8 mm that is increased to a densified sponge thickness of about 0.1 g/cm³ to about 0.5 g/cm³ by application of mechanical pressure.

15. A supple sponge structure according to claim 1, wherein the biocompatible hydrophilic polymer solution comprises a chitosan material.

16. A supple sponge structure according to claim 1, wherein the form is softened by a second softening device along a second axis.

17. A method comprising
providing a supple sponge structure as defined in claim 1, and
placing the supple sponge structure in contact with animal tissue.

18. A method according to claim 17
wherein placing the supple sponge structure in contact with animal tissue includes stuffing the supple sponge structure into a wound tract.

19. A method of making the supple sponge structure as defined in claim 1 comprising
providing a biocompatible hydrophilic polymer solution,
phase separating the hydrophilic polymer solution into the form by freezing from a room temperature to a freezing temperature at an overall cooling rate that is less than about 0.5° C./min, and
drying the form.

20. A method of making a supple sponge structure comprising
providing a biocompatible hydrophilic polymer solution,
phase separating the hydrophilic polymer solution into a form,
drying the form, and
softening the form by passage through a mechanical softening device comprising a plurality of spaced apart upper and lower rolling surfaces defining between them an undulating path sized and arranged to knead the form along an axis.

21. A method according to claim 20
wherein phase separating includes freezing from a room temperature to a freezing temperature at an overall cooling rate that is less than about 0.5° C./min.

22. A method according to claim 20
further including softening the form along a second axis different than the first defined axis.

23. A supple sponge structure adapted for placement in contact with animal tissue comprising a biocompatible hydrophilic polymer form, the form having been softened by a mechanical softening device comprising a plurality of spaced apart upper and lower rolling surfaces defining between them an undulating path sized and arranged to knead the form along an axis, the form being characterized by a suppleness that allows rolling the initial form upon itself without fracture into a roll form having a diameter less than either the width or the length.

24. A supple sponge structure according to claim 23, wherein the form has thickness of between 0.25 mm and about 4 mm.

25. A supple sponge structure according to claim 23, wherein the form has a thickness of about 0.9 mm.

26. A method comprising
providing a supple sponge structure as defined in claim 23, and
placing the supple sponge structure in contact with animal tissue.

27. A method according to claim 26
wherein placing the supple sponge structure in contact with animal tissue includes stuffing the supple sponge structure into a wound tract.

28. A method of treating an infection site involving Staphylococcus aureus bacteria and/or methicillin resistant Staphylococcus aureus bacteria comprising
providing a sponge structure comprising chitosan material,
placing the sponge structure on the infection site.
